(19) United States
(12) Patent Application Publication
(10) Pub. No.: US 2006/0019967 A1
(43) Pub. Date: Jan. 26, 2006

(54) SARS CoV main protease inhibitors

(76) Inventors: Su-Ying Wu, Tainan (TW); Hsing-Pang Hsieh, Taipei (TW); Tsu-An Hsu, Taipei (TW); I-Lin Lu, Kaohsiung (TW)

Correspondence Address: FISH & RICHARDSON PC, P.O. BOX 1022, MINNEAPOLIS, MN, 55440-1022, US

(21) Appl. No.: 11/185,917

(22) Filed: Jul. 20, 2005

Related U.S. Application Data
(60) Provisional application No. 60/589,685, filed on Jul. 21, 2004.

(51) Int. Cl. A61K 31/506; A61K 31/44; A61K 31/42; A61K 31/426; A61K 31/415

(52) U.S. Cl. 514/256; 514/357; 514/340; 514/341; 514/406; 514/378; 514/383; 514/365

(57) ABSTRACT

This invention relates to a method for modulating activity of SARS CoV main protease or an analogue thereof by contacting the protein with an effective amount of a compound of the following formula:
A₁-L-A₂
wherein A₁, A₂, and L are defined herein, and use of the compound in treating coronavirus infection, hepatitis C virus infection, hemophilia, vascular restenosis, or hypertension.

### CROSS REFERENCE TO RELATED APPLICATIONS

Pursuant to 35 USC § 119(e), this application claims priority to U.S. Provisional Application Ser. No. 60/589,685, filed Jul. 21, 2004, the contents of which are incorporated herein by reference.

### BACKGROUND

Proteins are the most abundant biological macromolecules, occurring in all cells and all parts of cells. They occur in great variety and exhibit enormous diversity of biological functions. From a set of 20 amino acids, widely diverse proteins are made to perform various biological effects in different organisms. Examples of proteins include enzymes, receptors, hormones, antibodies, and transporters.

A particular arrangement of amino acids may form binding sites in a protein, usually cavities on the protein surface or inside the protein. Ligands interact with a protein at its binding sites, causing a change of three dimensional shape of the protein. As a result, biological activities of the protein also change.

Proteins are important targets for drug development. The vast majority of successful therapies include the use of small molecule drugs that bind selectively to proteins and modulate (e.g., inhibit or promote) their activity.

### SUMMARY

This invention is based on the discovery that a group of dicyclic or multi-cyclic compounds effectively inhibit the activity of severe acute respiratory syndrome (SARS) coronavirus (CoV) main protease and hepatitis C virus (HCV) NS3 proteinase, a structural analogue of SARS CoV main protease.

One aspect of the present invention relates to a method for modulating the activity of a protein, i.e., SARS CoV main protease or its analogue by contacting the protein with an effective amount of a compound of Group I. The compound has the following formula:
A₁-L-A₂
wherein each of A₁ and A₂, independently, is phenyl, 5-membered heteroaryl, or 6-membered heteroaryl, optionally substituted with alkyl, haloalkyl, alkenyl, alkynyl, aryl, heteroaryl, halo, nitro, cyano, OR¹, OC(O)R¹, C(O)OR¹, C(O)R¹, SR¹, SO₂R¹, NR¹R², or NR¹C(O)R², or fused with a 3-8 membered ring; R¹ and R², independently, being H, alkyl, alkenyl, aryl, or heteroaryl; and L is —SO₂—, —C(R³R⁴)SO₂—, —C(R³R⁴)NR⁵—, —C(O)—, —C(O)S—, —C≡C—, —C(R³R⁴)C(O)O—, or —S(O)₂NR³—; each of R³, R⁴, and R⁵, independently, being H, alkyl, aryl, or heteroaryl.

The analogue of SARS CoV main protease refers to a protein having a structure in which the atomic coordinates of the C-alpha atoms have a root mean square deviation of not more than 2.3 Å, with respect to the corresponding C-alpha atoms of residue 1 to residue 189 of chain A of SARS CoV main protease. Examples include, but are not limited to, human coronavirus 229E main protease, transmissible gastroenteritis virus (TGEV) main protease, human chymase, human neutrophil elastase, human cathepsin, HCV NS3 proteinase, streptomyces griseus proteinase B, human coagulation factor Xa, alpha chymotrypsin, factor B serine protease, and collagenase.

In some embodiments, the compound modulates the activity of SARS CoV main protease or HCV NS3 proteinase. In other embodiments, the compound features that A₁ is phenyl, L is —SO₂—, or A₂ is phenyl.

Another aspect of this invention relates to a method for modulating the activity of a protein, i.e., SARS CoV main protease or its analogue by contacting the protein with an effective amount of a compound of Group II. The compound has the following formula:
A₁-L-A₂
wherein A₁ is 5-membered heteroaryl or 3 to 8-membered heterocyclyl, optionally substituted with alkyl, haloalkyl, alkenyl, alkynyl, aryl, heteroaryl, halo, nitro, cyano, OR¹, OC(O)R¹, C(O)OR¹, C(O)R¹, SR¹, SO₂R¹, NR¹R², or NR¹C(O)R², or fused with a 3-8 membered ring; R¹ and R², independently, being H, alkyl, alkenyl, aryl, or heteroaryl; A₂ is 5-membered heteroaryl, 6-membered heteroaryl, or 3 to 8-membered heterocyclyl, optionally substituted with alkyl, haloalkyl, alkenyl, alkynyl, aryl, heteroaryl, halo, nitro, cyano, OR³, OC(O)R³, C(O)OR³, C(O)R³, SR³, SO₂R³, NR³R⁴, or NR³C(O)R⁴, or fused with a 3 to 8-membered ring; R³ and R⁴, independently, being H, alkyl, alkenyl, aryl, or heteroaryl; and L is —SO₂—, —C(R⁵R⁶)S—, —C(R⁵R⁶)NR⁷—, —C(O)O—, —C(R⁵R⁶)C(O)O—, —C(R⁵R⁶)SO₂—, —C(O)NR⁵—, —C(O)—, —C(O)(CR⁵R⁶)—, —C(O)S—, —C≡C—, —O—, —S—, —N—, —C(S)NR⁵, or —SO₂NR⁵—; each of R⁵, R⁶, and R⁷, independently, being H, alkyl, aryl, or heteroaryl.

In some embodiments, the compound modulates the activity of SARS CoV main protease or HCV NS3 proteinase. In other embodiments, each of A₁ and A₂, independently, is 5-membered heteroaryl, such as triazolyl, pyrazolyl, thienyl, isoxazolyl, thiazolyl, furyl, or [1,3,4]oxadiazolyl.

Another aspect of this invention relates to a method for modulating the activity of a protein, i.e., SARS CoV main protease or its analogue by contacting the protein with an effective amount of a compound of Group III. The compound has the following formula:
A₁-L-A₂
wherein A₁ is phenyl optionally substituted with alkyl, haloalkyl, alkenyl, alkynyl, aryl, heteroaryl, halo, nitro, cyano, OR¹, OC(O)R¹, C(O)OR¹, C(O)R¹, SR¹, SO₂R¹, NR¹R², or NR¹C(O)R², or fused with a 3-8 membered ring; R¹ and R², independently, being H, alkyl, alkenyl, aryl, or heteroaryl; A₂ is 3 to 8-membered heterocyclyl, optionally substituted with alkyl, haloalkyl, alkenyl, alkynyl, aryl, heteroaryl, halo, nitro, cyano, OR³, OC(O)R³, C(O)OR³, C(O)R³, SR³, SO₂R³, NR³R⁴, or NR³C(O)R⁴, or fused with a 3 to 8-membered ring; R³ and R⁴, independently, being H, alkyl, alkenyl, aryl, or heteroaryl; and L is deleted, —C(R⁵R⁶)S—, —C(R⁵R⁶)NR⁷—, —C(O)O—, —C(R⁵R⁶)C(O)O—, —C(R⁵R⁶)SO₂—, —C(O)NR⁵—, —C(O)—, —C(O)(CR⁵R⁶)—, —C(O)S—, —C≡C—, —O—, —S—, —N—, —C(S)NR⁵, or —SO₂NR⁵—; each of R⁵, R⁶, and R⁷, independently, being H, alkyl, aryl, or heteroaryl.

Another aspect of this invention relates to a method for modulating the activity of a protein, i.e., SARS CoV main protease or its analogue by contacting the protein with an effective amount of a compound of Group IV. The compound has the following formula:
A₁-L-A₂
wherein each of A₁ and A₂, independently, is phenyl, or 5-membered heteroaryl, optionally substituted with alkyl, haloalkyl, alkenyl, alkynyl, aryl, heteroaryl, halo, nitro, cyano, OR¹, OC(O)R¹, C(O)OR¹, C(O)R¹, SR¹, SO₂R¹, NR¹R², or NR¹C(O)R², or fused with a 3-8 membered ring; R¹ and R², independently, being H, alkyl, alkenyl, aryl, or heteroaryl; and L is deleted, —SO₂—, —C(R³R⁴)SO₂—, —C(R³R⁴)NR⁵—, —C(O)—, —C(O)S—, —C≡C—, —C(R³R⁴)C(O)O—, or —S(O)₂NR³—; each of R³, R⁴, and R⁵, independently, being H, alkyl, aryl, or heteroaryl.

Another aspect of this invention relates to a method for treating coronavirus infection by administering to a subject in need thereof an effective amount of a compound of Group V. The compound has the following formula:
A₁-L-A₂
wherein each of A₁ and A₂, independently, is phenyl, 5-membered heteroaryl, or 6-membered heteroaryl, optionally substituted with alkyl, haloalkyl, alkenyl, alkynyl, aryl, heteroaryl, halo, nitro, cyano, OR¹, OC(O)R¹, C(O)OR¹, C(O)R¹, SR¹, SO₂R¹, NR¹R², or NR¹C(O)R², or fused with a 3-8 membered ring; R¹ and R², independently, being H, alkyl, alkenyl, aryl, or heteroaryl; and L is deleted, —SO₂—, —C(R³R⁴)SO₂—, —C(R³R⁴)NR⁵—, —C(O)—, —C(O)S—, —C≡C—, —C(R³R⁴)C(O)O—, or —S(O)₂NR³—; each of R³, R⁴, and R⁵, independently, being H, alkyl, aryl, or heteroaryl.

In some embodiments, the coronovirus infection is SARS virus infection or TEGV infection. In other embodiments, A₁ is phenyl, L is —SO₂—, or A₂ is phenyl, pyrimidinyl, pyrazolyl, or pyridinyl.

Another aspect of this invention relates to a method for treating coronavirus infection by administering to a subject in need thereof an effective amount of a compound of Group VI. The compound has the following formula:
A₁-L-A₂
wherein A₁ is phenyl, 5-membered heteroaryl, or 3 to 8-membered heterocyclyl, optionally substituted with alkyl, haloalkyl, alkenyl, alkynyl, aryl, heteroaryl, halo, nitro, cyano, OR¹, OC(O)R¹, C(O)OR¹, C(O)R¹, SR¹, SO₂R¹, NR¹R², or NR¹C(O)R², or fused with a 3-8 membered ring; R¹ and R², independently, being H, alkyl, alkenyl, aryl, or heteroaryl; A₂ is 5-membered heteroaryl, 6-membered heteroaryl, or 3 to 8-membered heterocyclyl, optionally substituted with alkyl, haloalkyl, alkenyl, alkynyl, aryl, heteroaryl, halo, nitro, cyano, OR³, OC(O)R³, C(O)OR³, C(O)R³, SR³, SO₂R³, NR³R⁴, or NR³C(O)R⁴, or fused with a 3 to 8-membered ring; R³ and R⁴, independently, being H, alkyl, alkenyl, aryl, or heteroaryl; and L is deleted, —SO₂—, —C(R⁵R⁶)S—, —C(R⁵R⁶)NR⁷—, —C(O)O—, —C(R⁵R⁶)C(O)O—, —C(R⁵R⁶)SO₂—, —C(O)NR⁵—, —C(O)—, —C(O)(CR⁵R⁶)—, —C(O)S—, —C≡C—, —O—, —S—, —N—, or —SO₂NR⁵—; each of R⁵, R⁶, and R⁷, independently, being H, alkyl, aryl, or heteroaryl.

In some embodiments, the coronovirus infection is SARS virus infection or TEGV infection. In other embodiments, each of A₁ and A₂, independently, is 5-membered heteroaryl, such as triazolyl, pyrazolyl, thienyl, isoxazolyl, thiazolyl, furyl, or [1,3,4]oxadiazolyl.

Another aspect of this invention relates to a method for treating hepatitis C virus infection by administering to a subject in need thereof an effective amount of a compound of Group VII. The compound has the following formula:
A₁-L-A₂
wherein each of A₁ and A₂, independently, is phenyl, 5-membered hetereoaryl, or 3 to 8-membered heterocyclyl, each of which is optionally substituted with alkyl, haloalkyl, alkenyl, alkynyl, aryl, heteroaryl, halo, carboxy, acylalkyl, nitro, cyano, OR¹, OC(O)R¹, C(O)OR¹, C(O)R¹, SR¹, SO₂R¹, NR¹R², or NR¹C(O)R², or fused with a 3-8 membered ring; R¹ and R², independently, being H, alkyl, alkenyl, aryl, or heteroaryl; and L is deleted, —SO₂—, —C(R³R⁴)S—, —C(R³R⁴)NR⁵—, —C(O)O—, —C(R³R⁴)C(O)O—, —C(R³R⁴)SO₂—, —C(O)NR³—, C(R³R⁴)NR⁵, —C(O)—, —C(O)(CR³R⁴)—, —C(O)S—, —C≡C—, —O—, —S—, —N—, —C(S)NR³, or —SO₂NR⁵—; each of R³, R⁴, and R⁵, independently, being H, alkyl, aryl, or heteroaryl.

Another aspect of this invention relates to a method for treating hepatitis C virus infection by administering to a subject in need thereof an effective amount of a compound of Group VIII. The compound has the following formula:
A₁-L-A₂
wherein each of A₁ and A₂, independently, is phenyl, 5-membered hetereoaryl, 6-membered heteroaryl, or 3 to 8-membered heterocyclyl, each of which is optionally substituted with alkyl, haloalkyl, alkenyl, alkynyl, aryl, heteroaryl, halo, carboxy, acylalkyl, nitro, cyano, OR¹, OC(O)R¹, C(O)OR¹, C(O)R¹, SR¹, SO₂R¹, NR¹R², or NR¹C(O)R², or fused with a 3-8 membered ring; R¹ and R², independently, being H, alkyl, alkenyl, aryl, or heteroaryl; and L is deleted, —SO₂—, —C(R³R⁴)S—, —C(R³R⁴)NR⁵—, —C(O)O—, —C(R³R⁴)C(O)O—, —C(R³R⁴)SO₂—, —C(O)NR³—, —C(O)—, —C(O)(CR³R⁴)—, —C(O)S—, —C≡C—, —O—, —S—, —N—, —C(S)NR³, or —SO₂NR⁵—; each of R³, R⁴, and R⁵, independently, being H, alkyl, aryl, or heteroaryl.

Another aspect of this invention relates to a method for treating hemophilia by administering to a subject in need thereof an effective amount of a compound Group IX. The compound has the following formula:
A₁-L-A₂
wherein each of A₁ and A₂, independently, is phenyl, 5-membered hetereoaryl, or 6-membered heteroaryl, or 3 to 8-membered heterocyclyl, each of which is optionally substituted with alkyl, haloalkyl, alkenyl, alkynyl, aryl, heteroaryl, halo, carboxy, acylalkyl, nitro, cyano, OR¹, OC(O)R¹, C(O)OR¹, C(O)R¹, SR¹, SO₂R¹, NR¹R², or NR¹C(O)R², or fused with a 3-8 membered ring; R¹ and R², independently, being H, alkyl, alkenyl, aryl, or heteroaryl; and L is deleted, —SO₂—, —C(R³R⁴)S—, —C(R³R⁴)NR⁵—, —C(O)O—, —C(R³R⁴)C(O)O—, —C(R³R⁴)SO₂—, —C(O)NR³—, C(R³R⁴)NR⁵, —C(O)—, —C(O)(CR³R⁴)—, —C(O)S—, —C≡C—, —O—, —S—, —N—, —C(S)NR³, or —SO₂NR⁵—; each of R³, R⁴, and R⁵, independently, being H, alkyl, aryl, or heteroaryl.

Still another aspect of this invention relates to a method for treating vascular restenosis or hypertension by administering to a subject in need thereof an effective amount of a compound of Group X. The compound has the following formula:
A₁-L-A₂
wherein each of A₁ and A₂, independently, is phenyl, 5-membered heteroaryl, or 6 six-membered heteroaryl, each of which is optionally substituted with alkyl, haloalkyl, alkenyl, alkynyl, aryl, heteroaryl, halo, carboxy, acylalkyl, nitro, cyano, OR¹, OC(O)R¹, C(O)OR¹, C(O)R¹, SR¹, SO₂R¹, NR¹R², or NR¹C(O)R², or fused with a 3-8 membered ring; R¹ and R², independently, being H, alkyl, alkenyl, aryl, or heteroaryl; and L is deleted, —C(R³R⁴)S—, —C(R³R⁴)NR⁵—, —C(O)O—, —C(R³R⁴)C(O)O—, —C(R³R⁴)SO₂—, —C(O)NR⁵—, C(R³R⁴)NR⁵, —C(O)—, —C(O)(CR³R⁴)—, —C(O)S—, —SO₂—, —C≡C—, —O—, —S—, —N—, or —SO₂NR⁵—; each of R³, R⁴, and R⁵, independently, being H, alkyl, aryl, or heteroaryl.

Shown below are exemplary compounds used in each of the above-described methods:

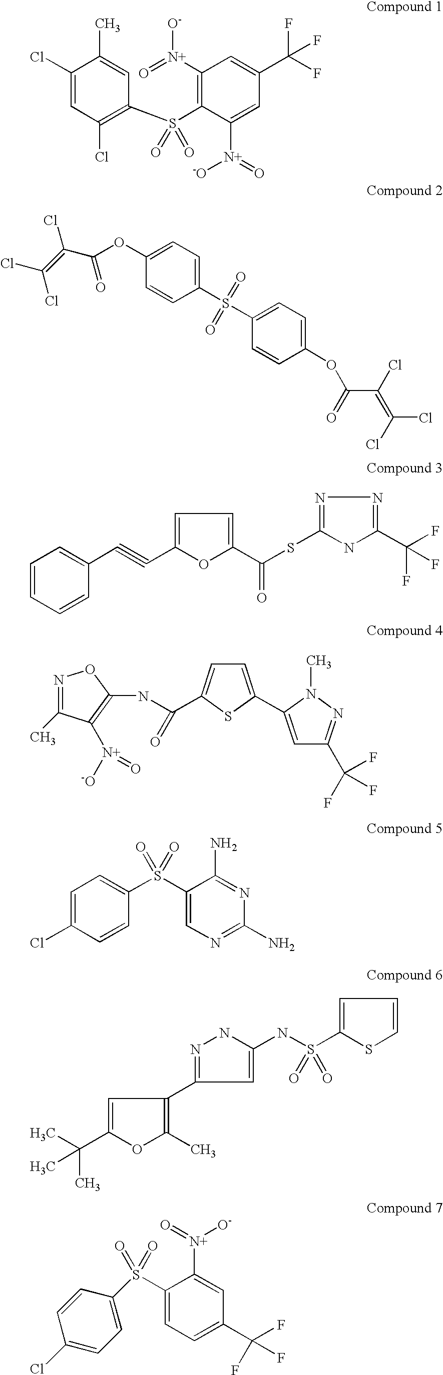

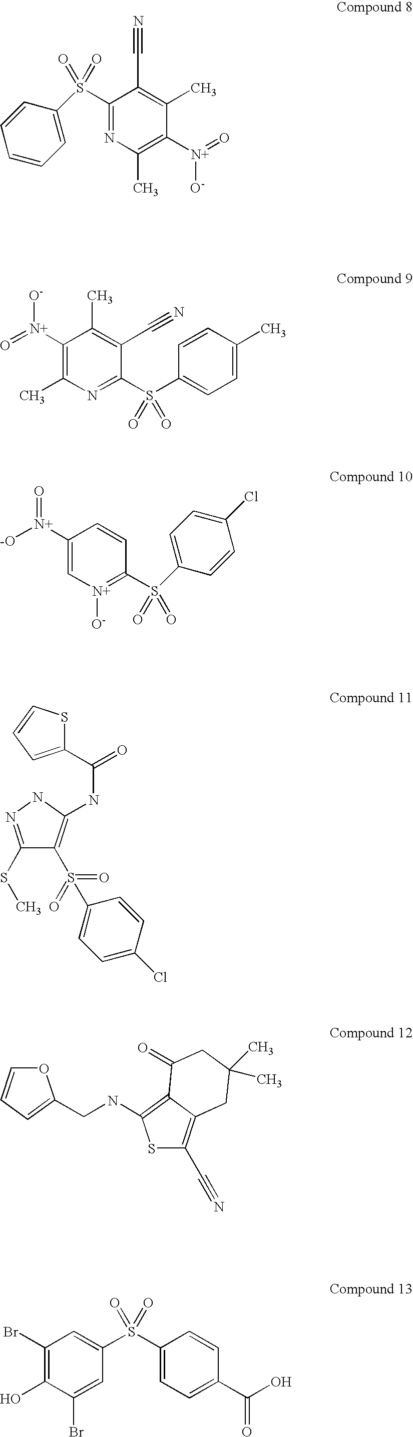

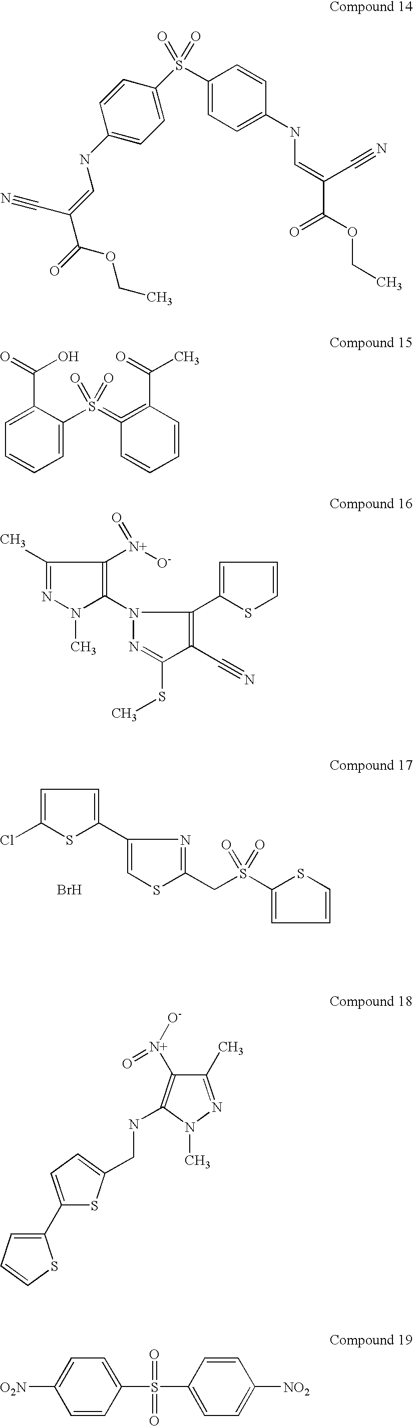

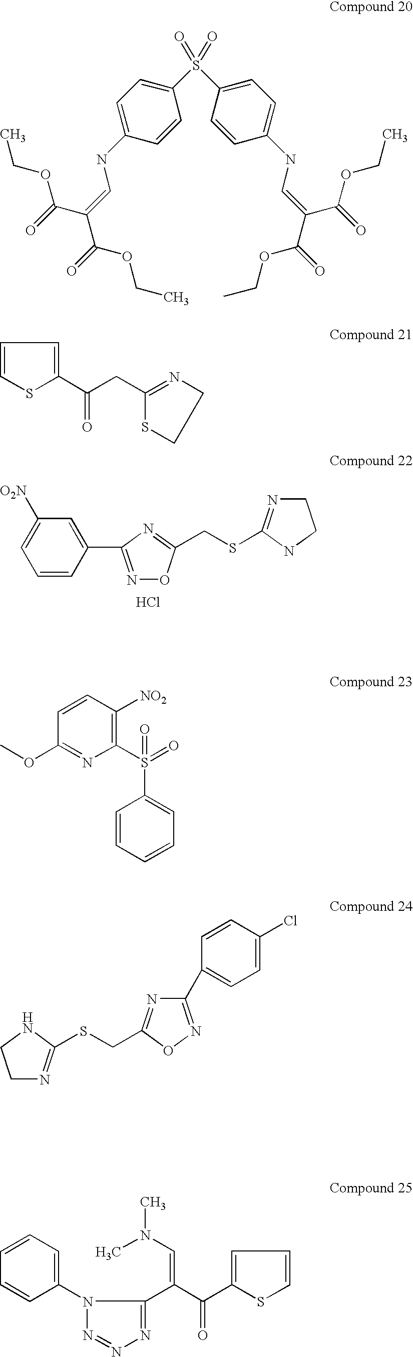

The term “alkyl” refers to a straight or branched hydrocarbon, containing 1-10 carbon atoms. Examples of alkyl groups include, but are not limited to, methyl, ethyl, n-propyl, i-propyl, n-butyl, i-butyl, and t-butyl. The term “haloalkyl” refers to alkyl substituted with one or more halo groups.

The term “alkenyl” refers to a straight or branched hydrocarbon having one or more carbon-carbon double bonds. The term “alkynyl” refers to a straight or branched hydrocarbon having one or more carbon-carbon triple bonds.

The term “aryl” refers to a 6-carbon monocyclic, 10-carbon bicyclic, 14-carbon tricyclic aromatic ring system wherein each ring may have 1 to 4 substituents. Examples of aryl groups include, but are not limited to, phenyl, naphthyl, and anthracenyl.

The term “cyclyl” refers to a saturated and partially unsaturated cyclic hydrocarbon group having 3 to 12 carbons, preferably 3 to 8 carbons, and more preferably 3 to 6 carbons, wherein the cyclyl group may be optionally substituted. Examples of cyclyl groups include, without limitation, cyclopropyl, cyclobutyl, cyclopentyl, cyclopentenyl, cyclohexyl, cyclohexenyl, cycloheptyl, and cyclooctyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein each ring may have 1 to 4 substituents. Examples of heteroaryl groups include pyridyl, furyl or furanyl, imidazolyl, benzimidazolyl, pyrimidinyl, thiophenyl or thienyl, quinolinyl, indolyl, thiazolyl, and the like.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, and the like.

Alkyl, haloalkyl, alkenyl, alkynyl, aryl, and heteroaryl mentioned herein include both substituted and unsubstituted moieties. Examples of a substituent include, but are not limited to, halo, hydroxyl, amino, cyano, nitro, mercapto, alkoxycarbonyl, amido, carboxy, alkanesulfonyl, alkylcarbonyl, carbamido, carbamyl, carboxyl, thioureido, thiocyanato, sulfonamido, alkyl, alkenyl, alkynyl, alkyloxy, aryl, heteroaryl, cyclyl, heterocyclyl, in which alkyl, alkenyl, alkynyl, alkyloxy, aryl, heteroaryl cyclyl, and heterocyclyl may be further substituted.

Also within the scope of this invention are (1) a composition containing a compound of Group V or VI and a pharmaceutically acceptable carrier for use in treating coronavirus infection, as well as the use of such a composition for the manufacture of a medicament for the infection; (2) a composition containing a compound of Group VII or VIII and a pharmaceutically acceptable carrier for use in treating hepatitis C virus infection, as well as the use of such a composition for the manufacture of a medicament for treating the infection; (3) a composition containing a compound of Group IX and a pharmaceutically acceptable carrier for use in treating hemophilia, as well as the use of such a composition for the manufacture of a medicament for treating hemophilia; and (4) a composition containing a compound of Group X and a pharmaceutically acceptable carrier for use in treating vascular restenosis or hypertension, as well as the use of such a composition for the manufacture of a medicament for treating vascular restenosis or hypertension

The details of many embodiments of the invention are set forth in the description below. Other features, objects, and advantages of the invention will be apparent from the description and the claims.

### DESCRIPTION OF DRAWINGS

FIG. 1 lists the atomic structure coordinates for SARS CoV main protease crystal in complex with an inhibitor, Cbz-Val-Asn-Ser-Thr-Leu-Cln-CMK. “X, Y, Z” crystallographically define the atomic position determined for each atom. “Occ” is an occupancy factor that refers to the fraction of the molecules in which each atom occupies the position specified by the coordinates. A value of “1.00” indicates that each atom has the same conformation, i.e., the same position, in all molecules of the crystal. “B” is a thermal factor that measures movement of the atom around its atomic center.

### DETAILED DESCRIPTION

The compounds used to practice this invention can be prepared by methods well known in the art. For example, a substituted aryl ring is reacted with a substituted aryl sulfonyl chloride in the presence of a Friedel-Craft catalyst to a diaryl sulfone compound. As another example, a chloro-substituted aryl or heteroaryl ring is reacted with another aryl or heteroaryl ring in the presence of a palladium catalyst to form a directly-linked compound. Synthetic chemistry transformations and protecting group methodologies (protection and deprotection) useful in preparing the compounds include, e.g., those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The compounds mentioned above may contain one or more asymmetric centers. Thus, they occur as racemates and racemic mixtures, single enantiomers, individual diastereomers, diastereomeric mixtures, or cis- or trans-isomeric forms. All such isomeric forms are contemplated.

This invention features a method for modulating the activity of SARS CoV main protease or an analogue thereof using an effective amount of a compound of Group I, II, III, or IV shown above. The term “an effective amount” refers to the amount of the compound that is required to confer the above-described effect on the subject. The effective amount varies, as recognized by those skilled in the art, depending on the type of the effect, route of administration, excipient usage, and the possibility of co-usage with other treatment. An analogue of SARS CoV main protease, as mentioned above, is a protein having a structure in which the atomic coordinates of the C-alpha atoms have a root mean square deviation of not more than 2.3 Å, with respect to the corresponding C-alpha atoms of residue 1 to residue 189 of chain A of SARS CoV main protease of SARS CoV main protease. Examples include, but are not limited to, human coronavirus 229E main protease, TGEV main protease, human chymase, human neutrophil elastase, human cathepsin, HCV NS3 proteinase, streptomyces griseus proteinase B, human coagulation factor Xa, alpha chymotrypsin, factor B serine protease, or collagenase. The term “root mean square deviation” refers to the square root of the arithmetic mean of the squares of the deviation from the mean. For purposes of this invention, the root mean square deviation is calculated, after optimal superposition of SARS CoV main protease and a potential analogue thereof, as the square root of the mean square distances between C-alpha atoms of residue 1 to residue 189 of chain A of SARS CoV main protease and the corresponding C-alpha atoms in the backbones of the potential analogue.

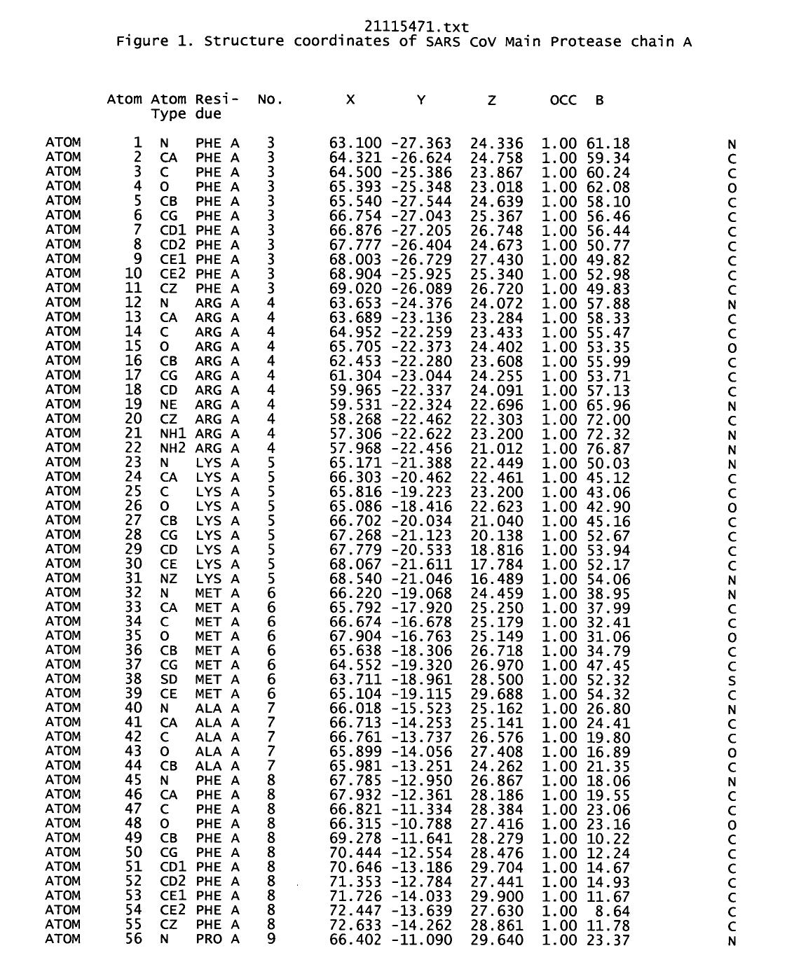

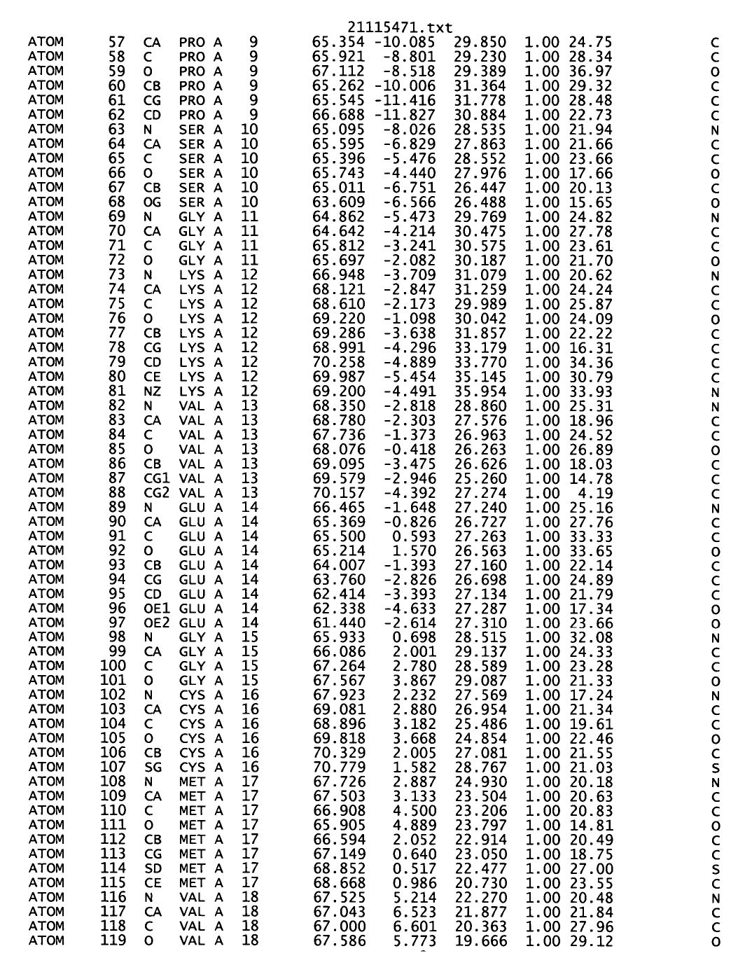

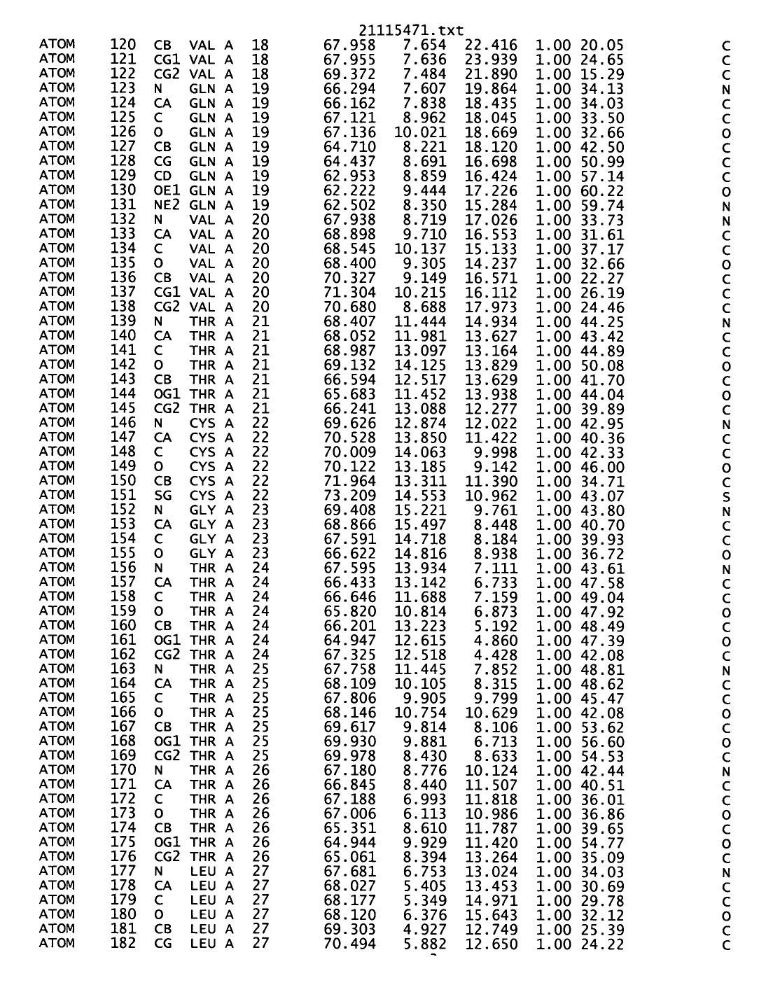

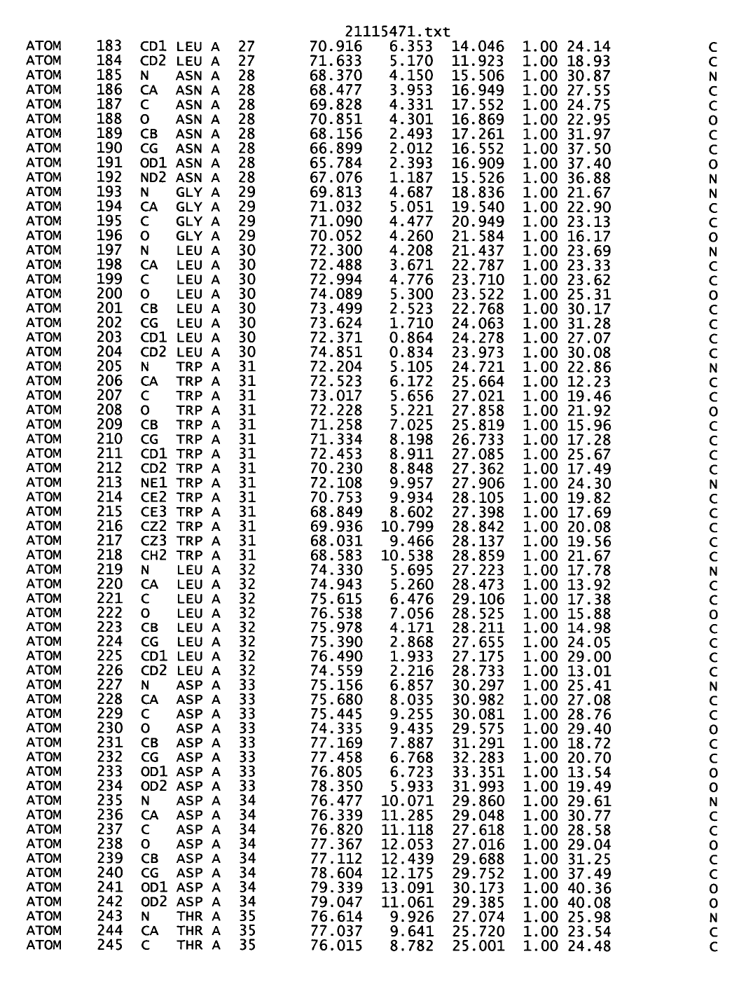

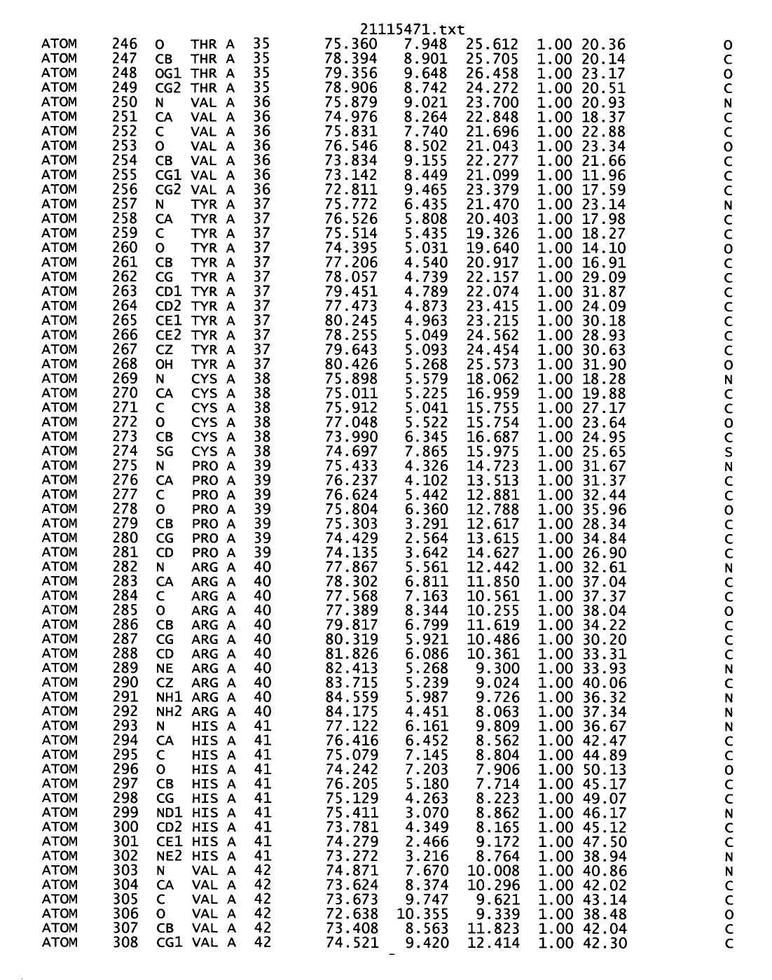

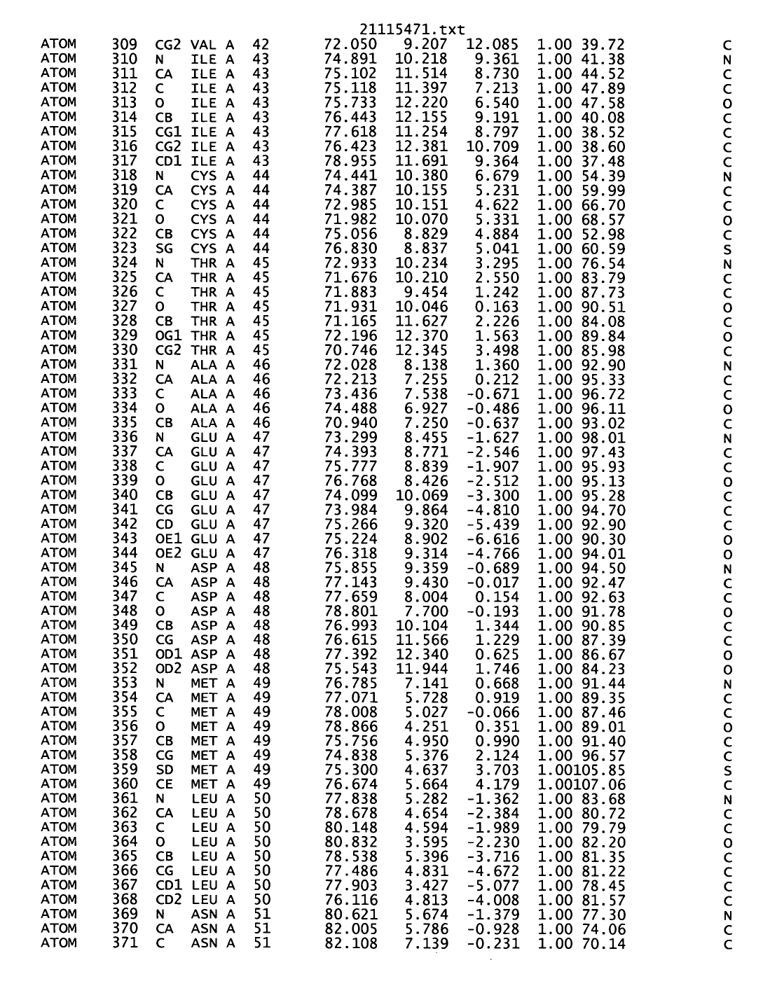

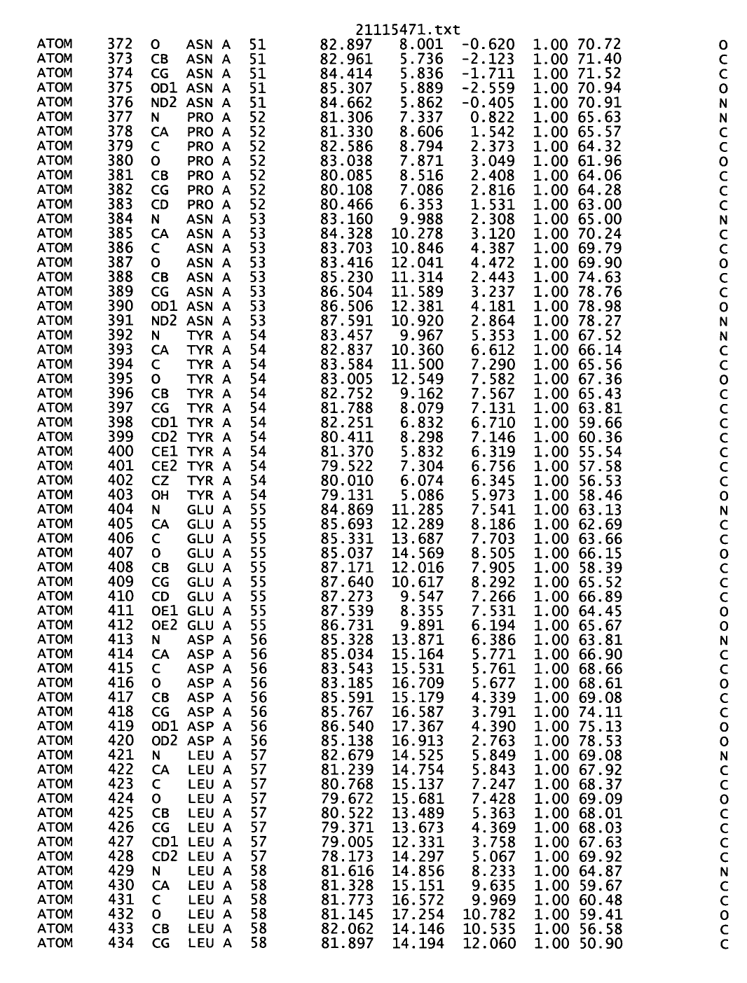

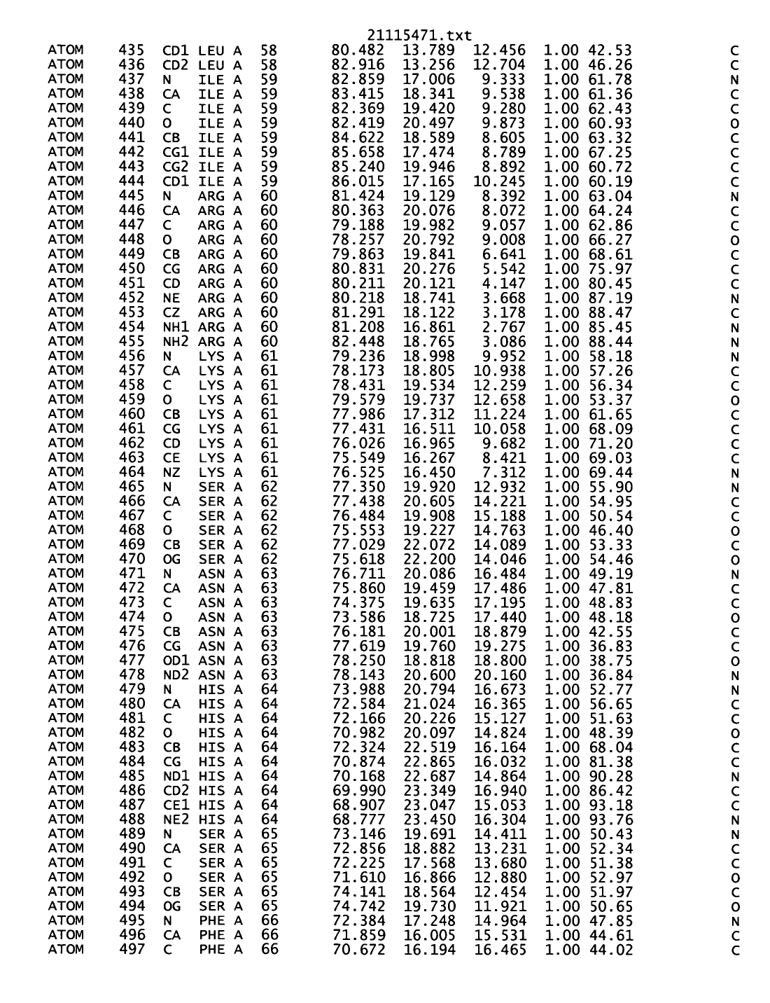

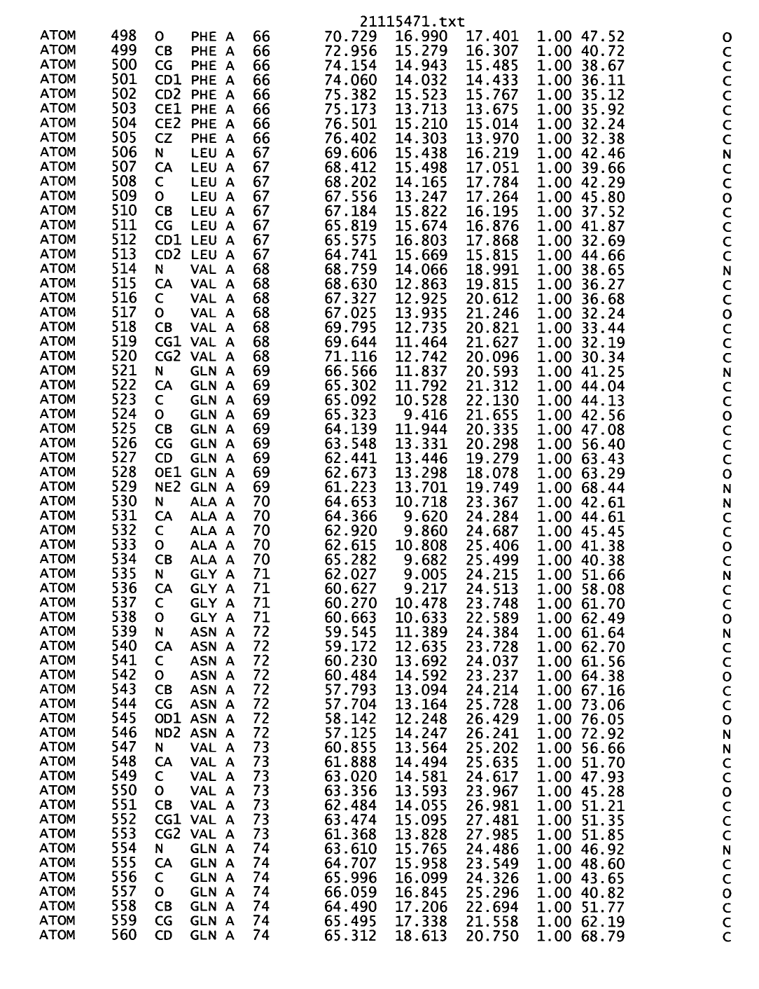

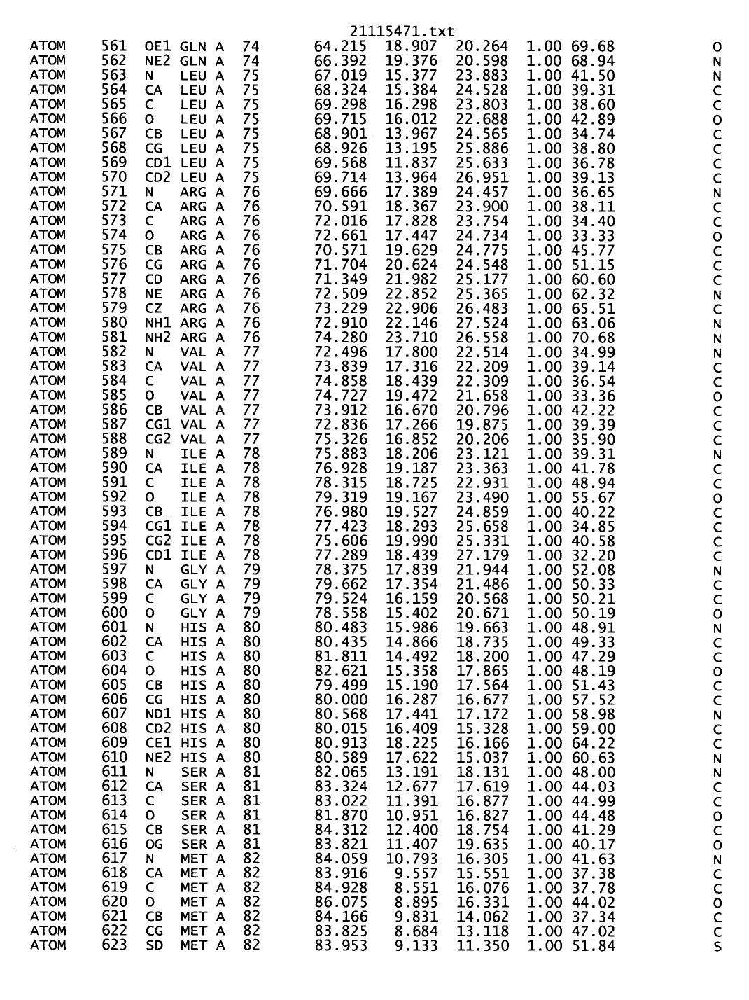

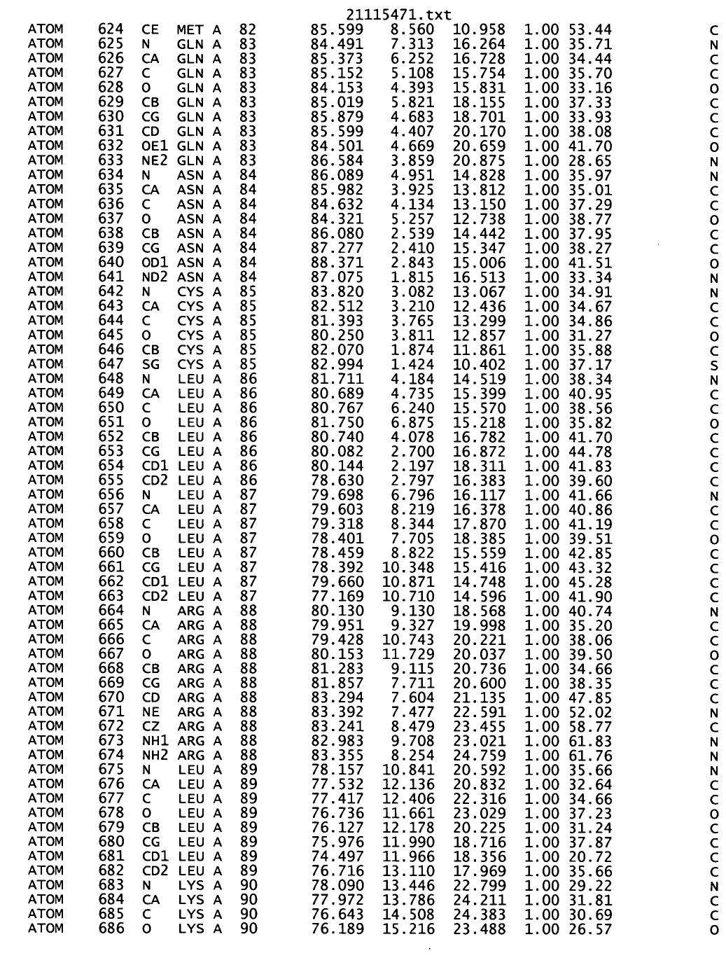

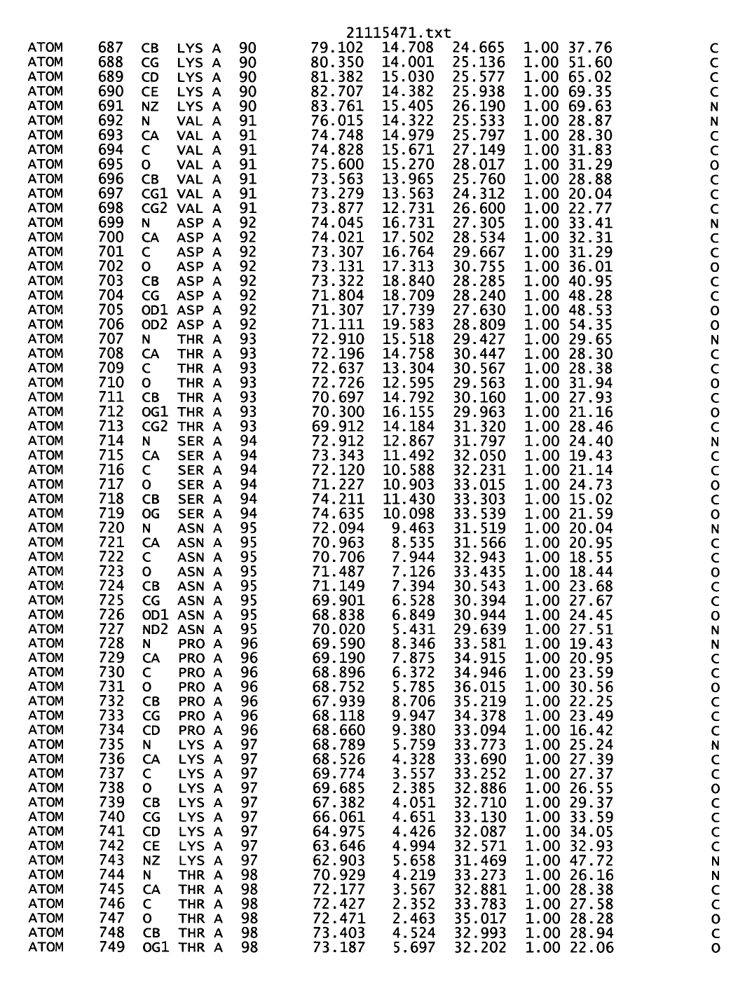

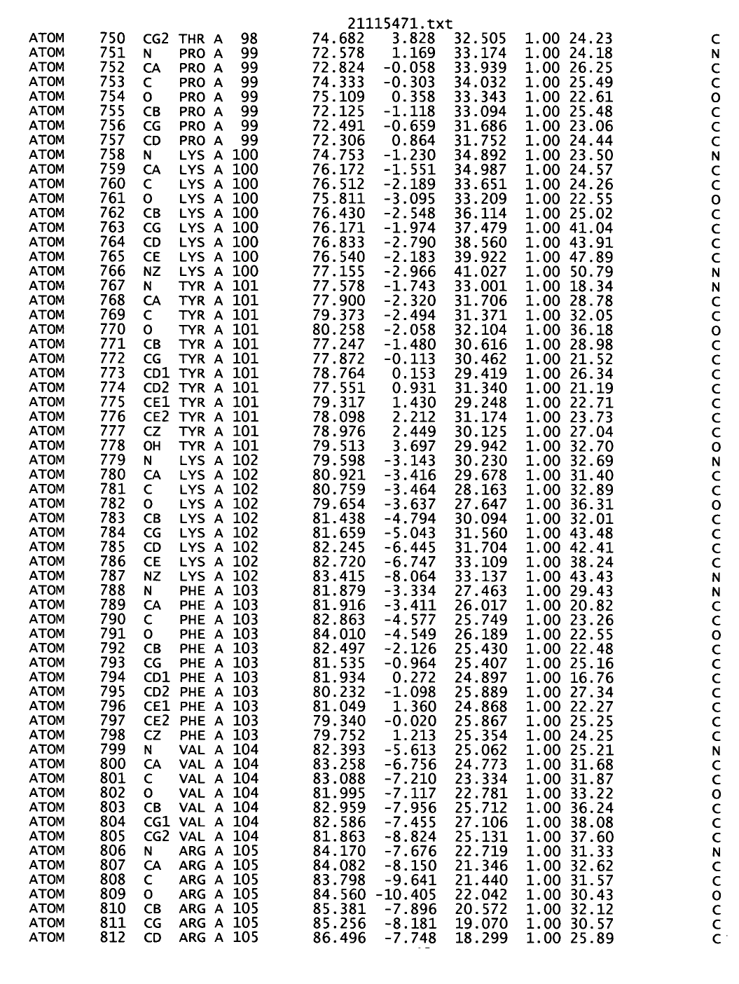

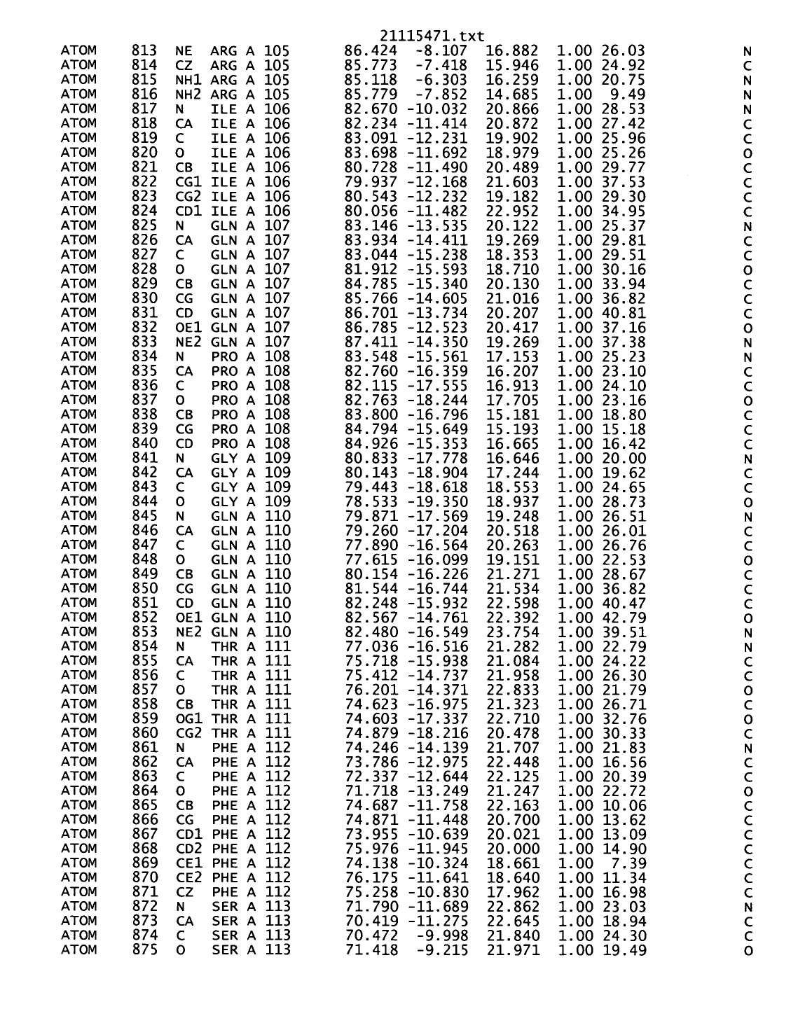

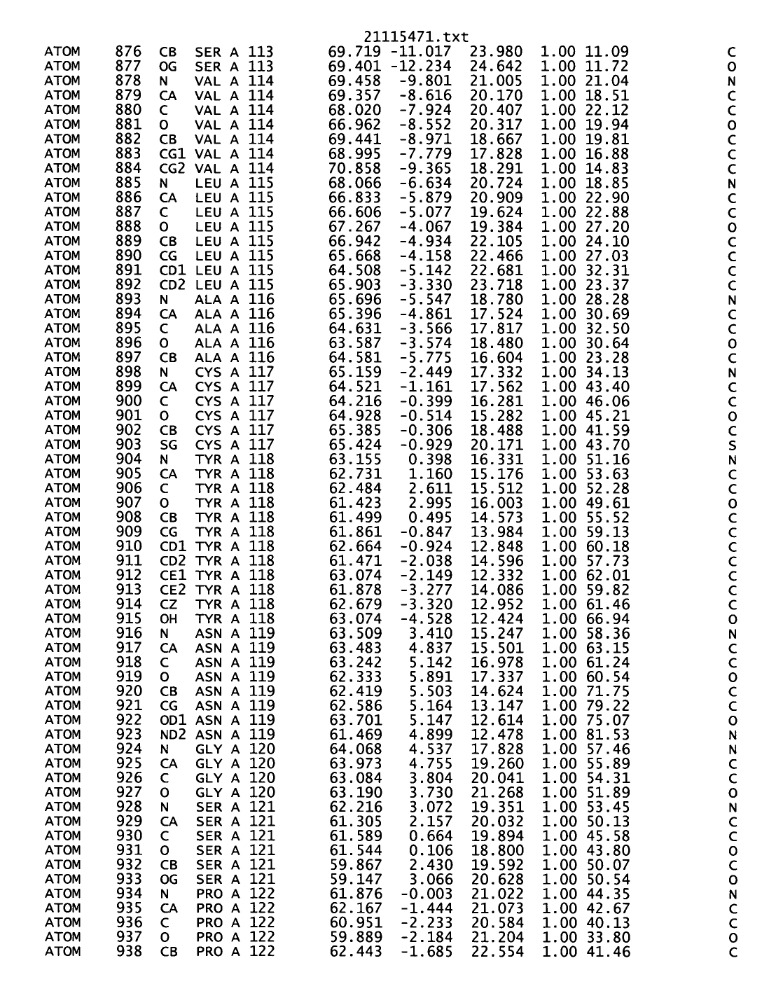

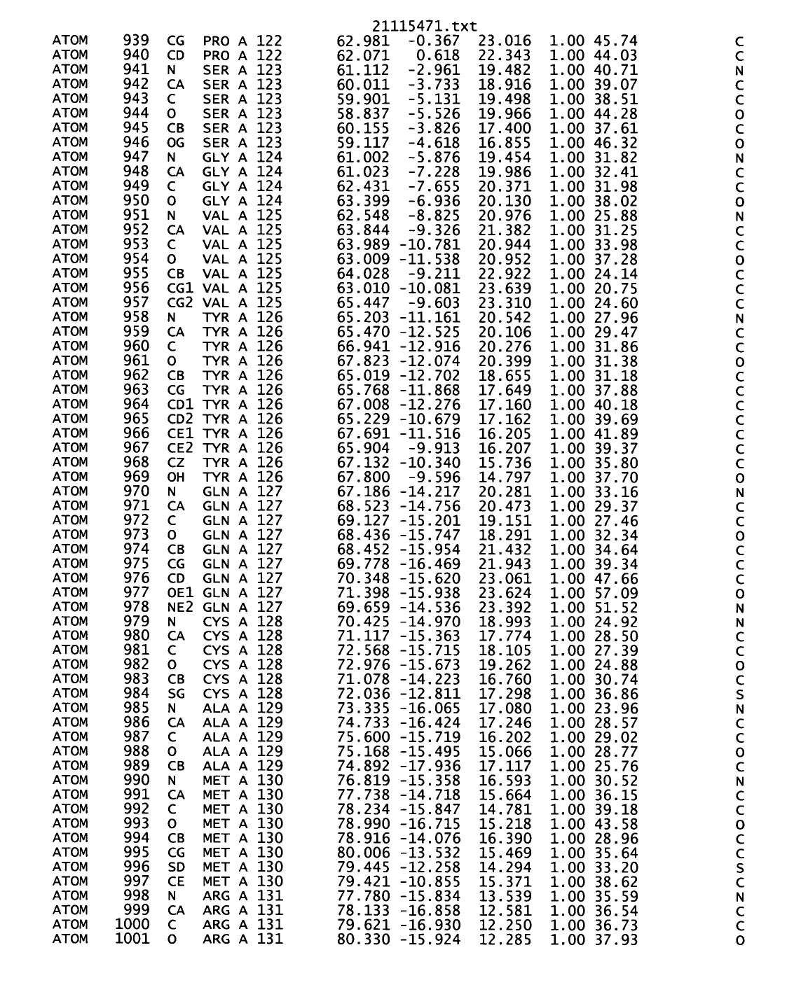

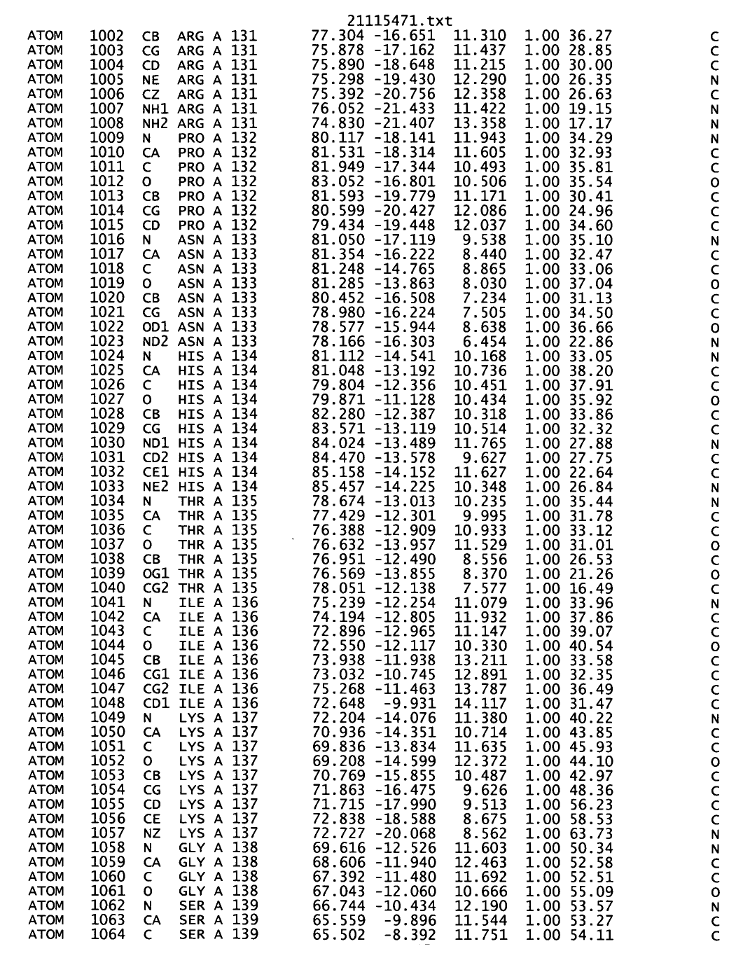

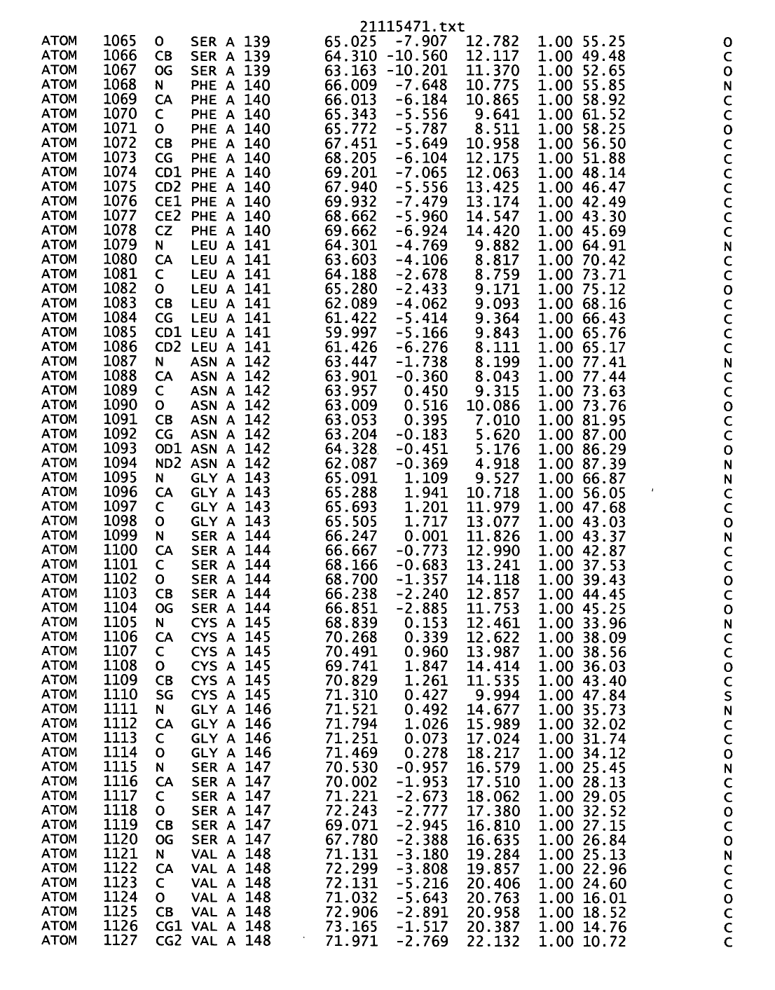

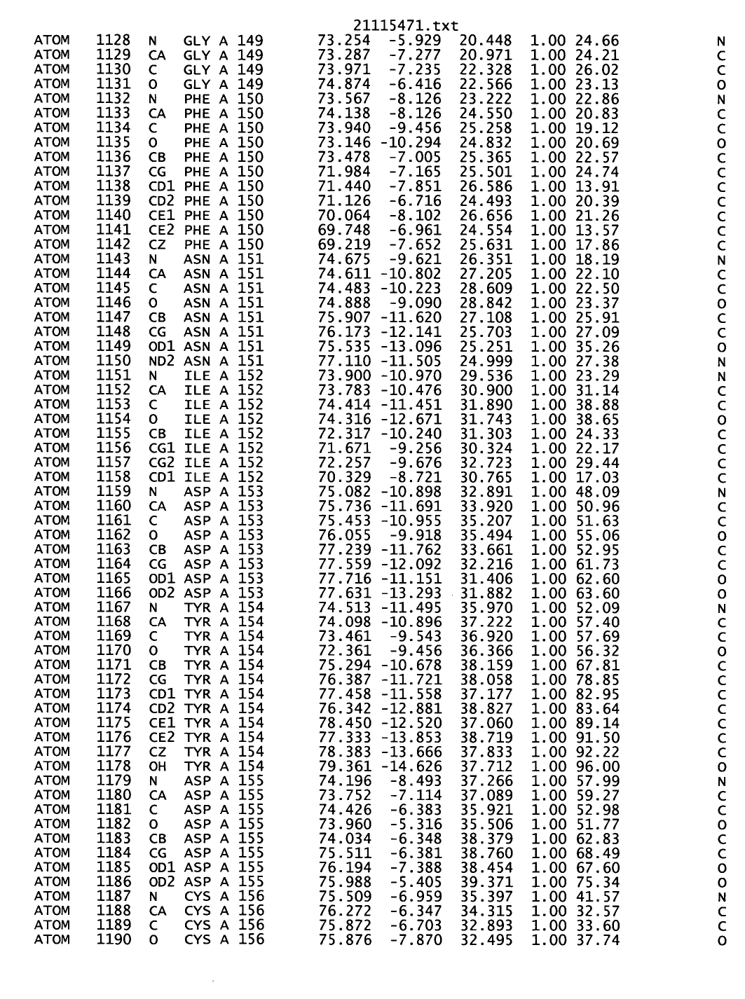

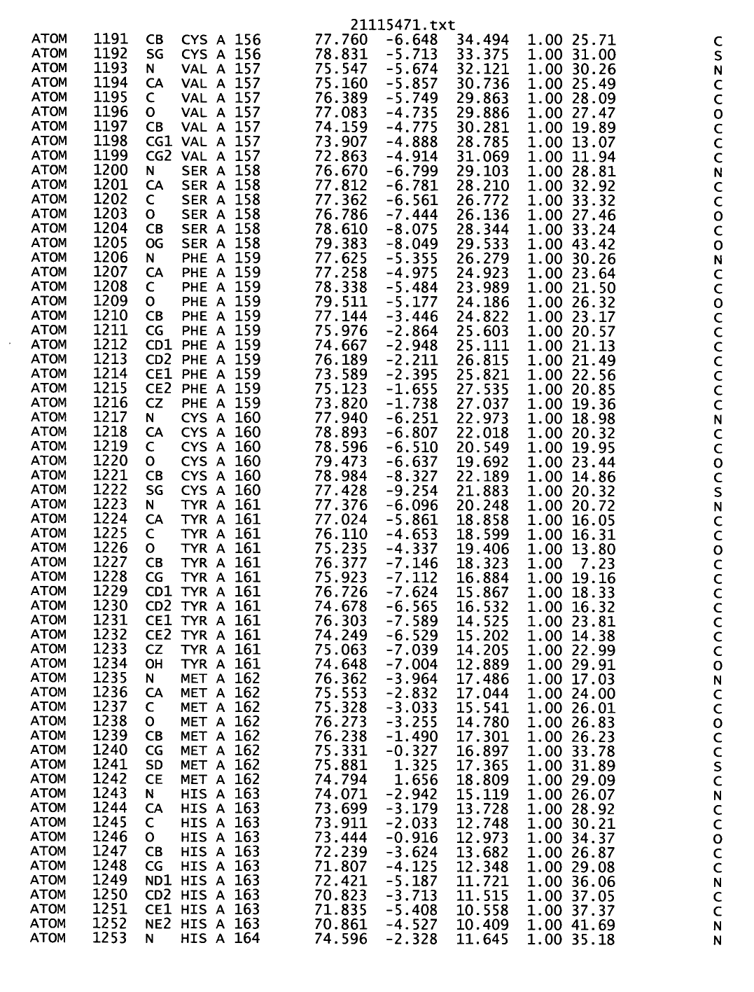

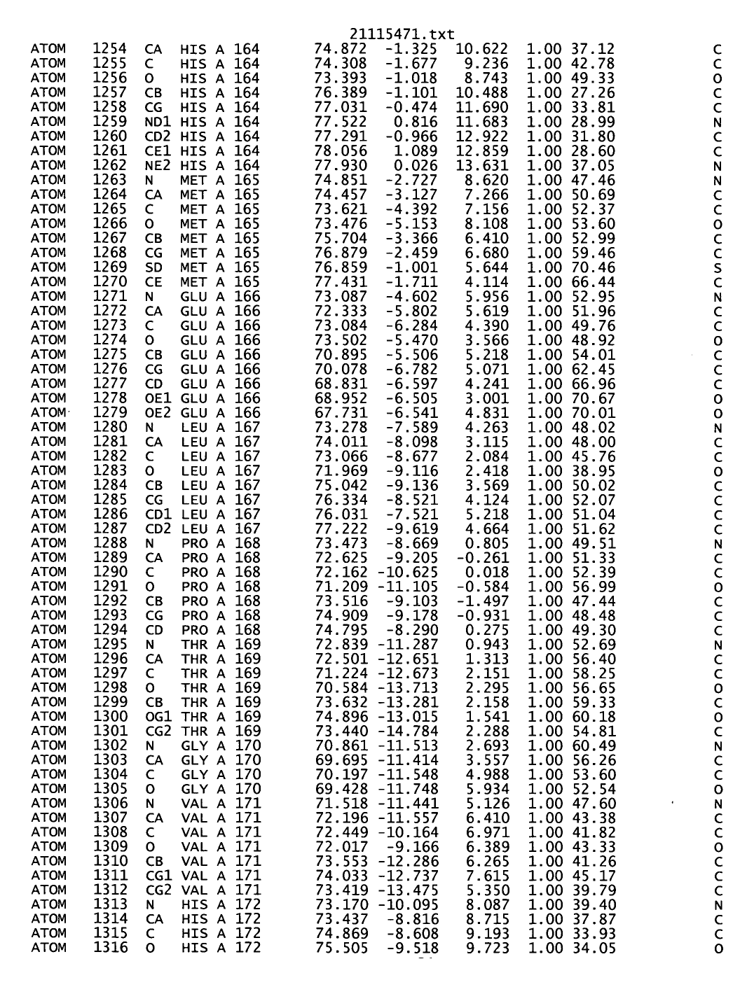

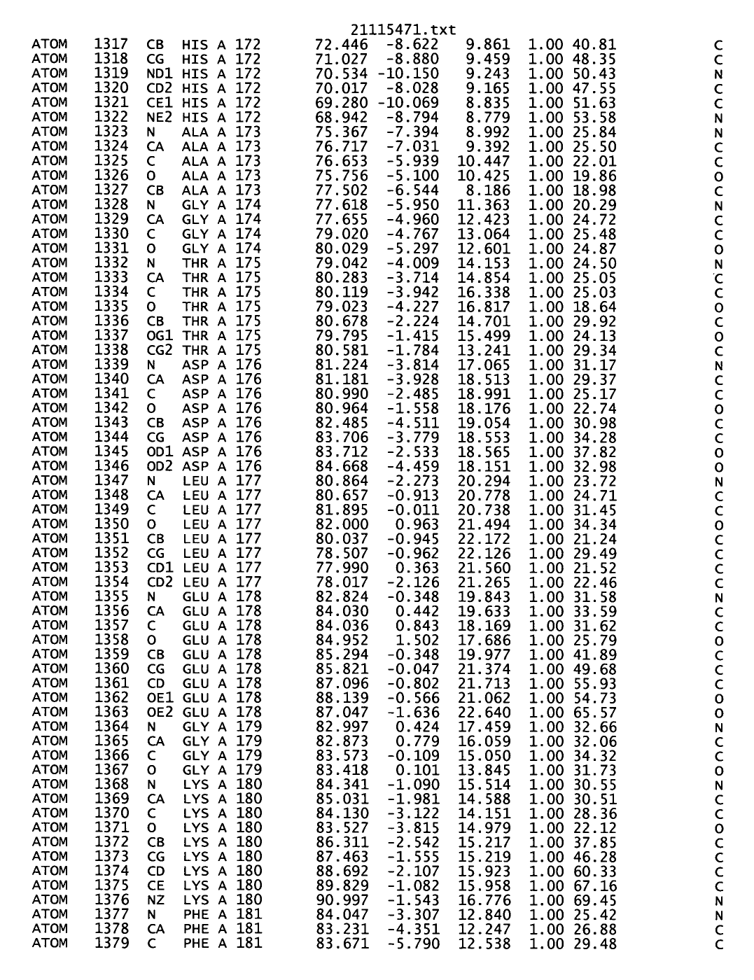

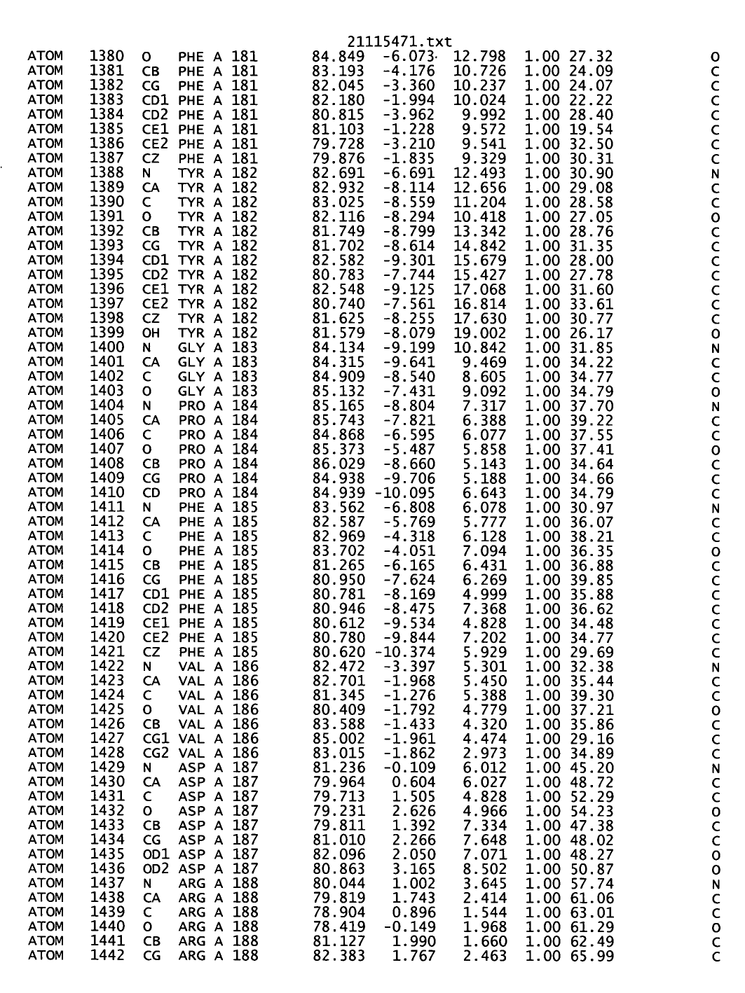

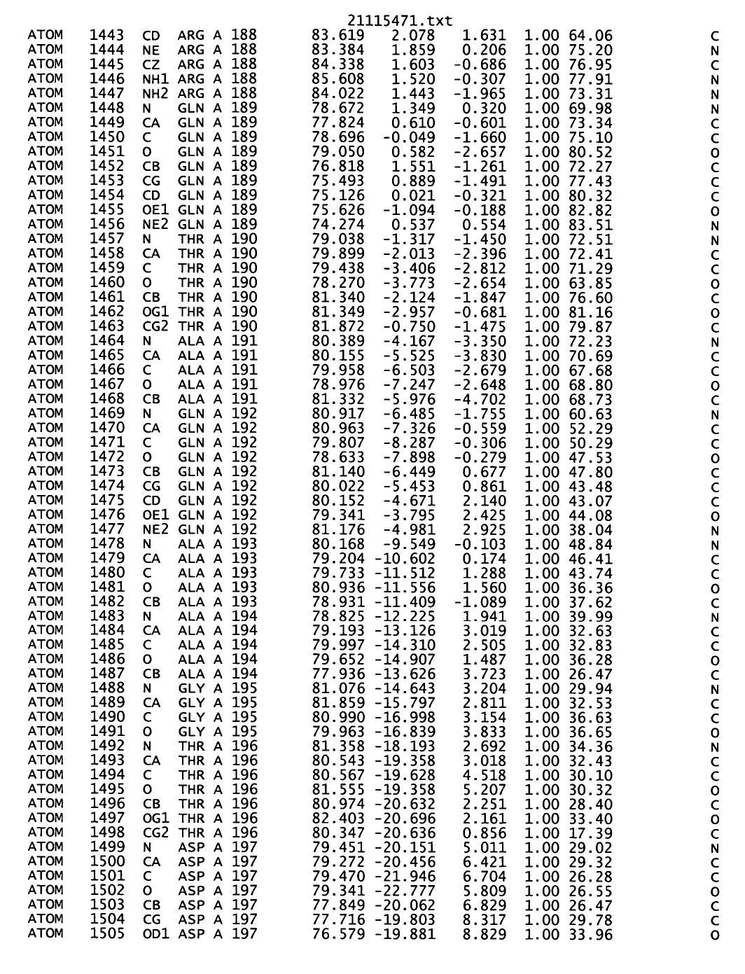

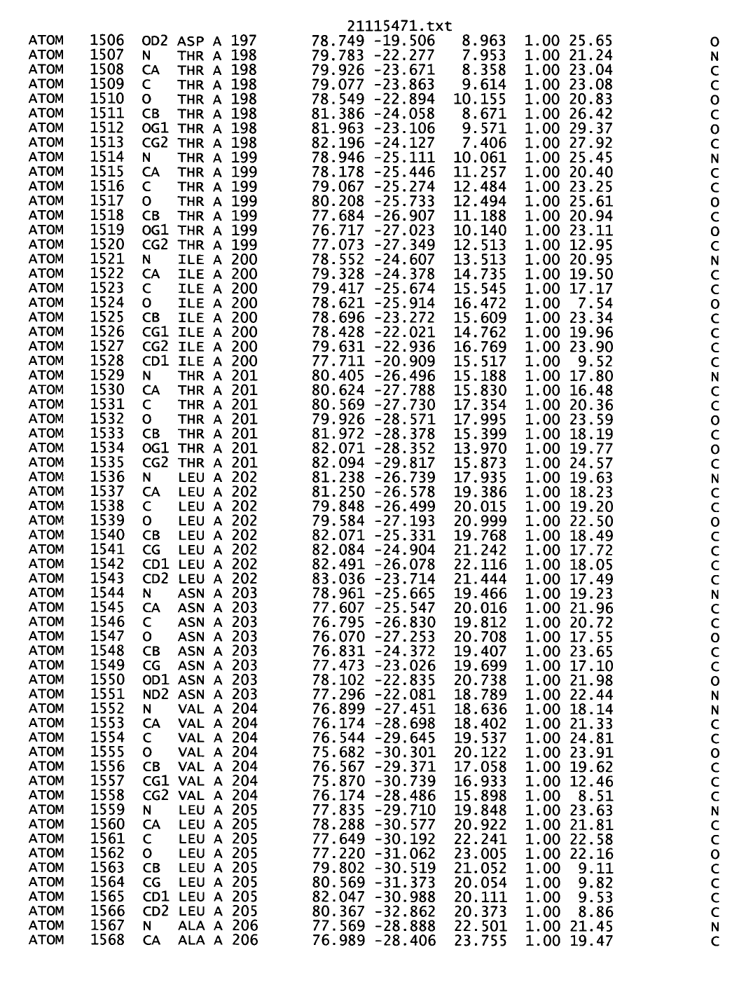

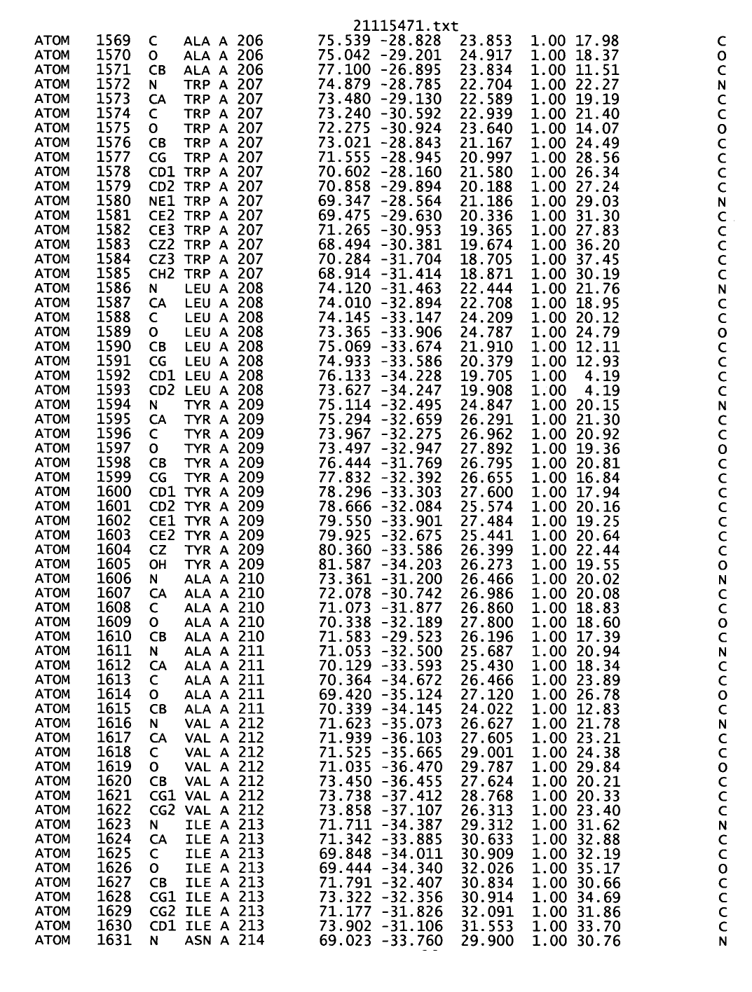

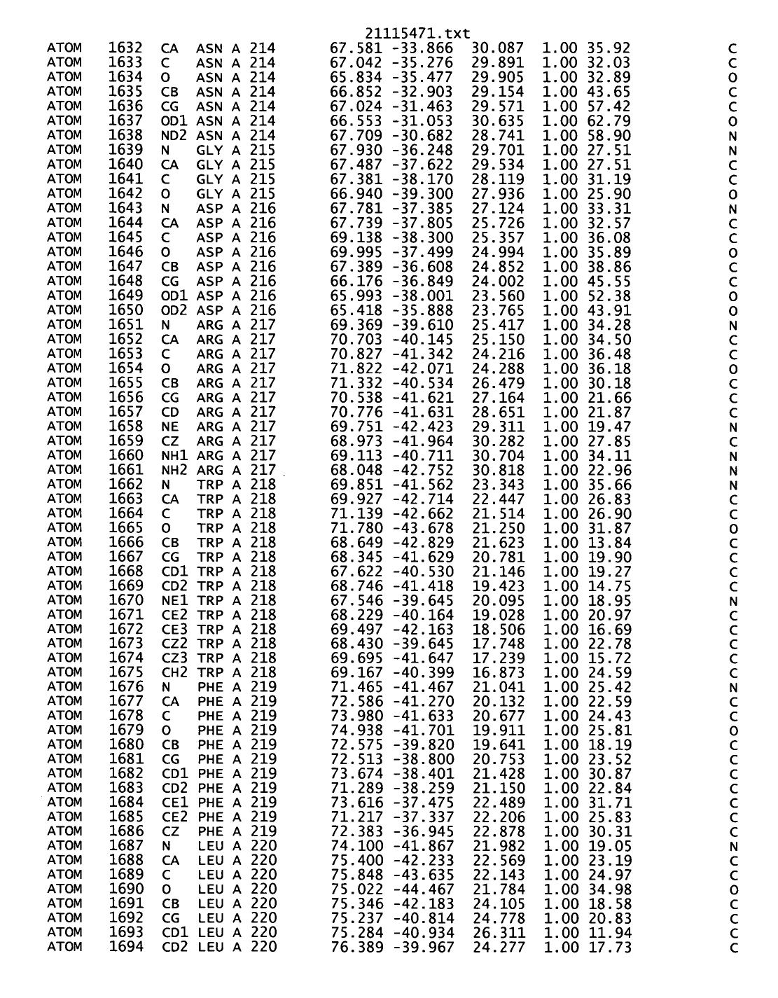

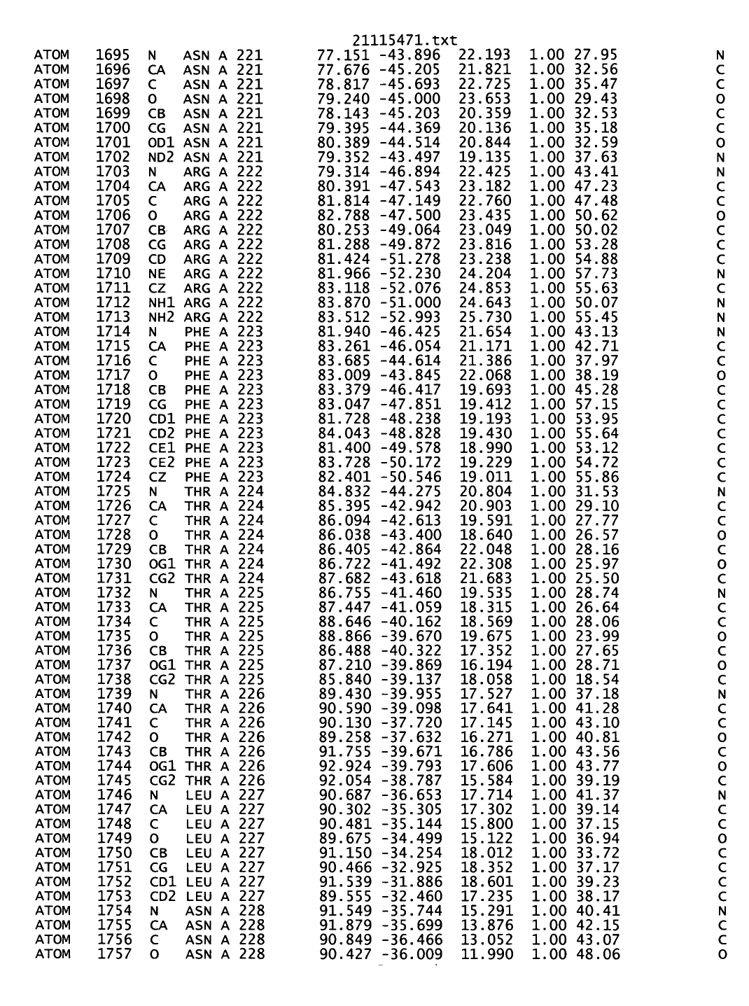

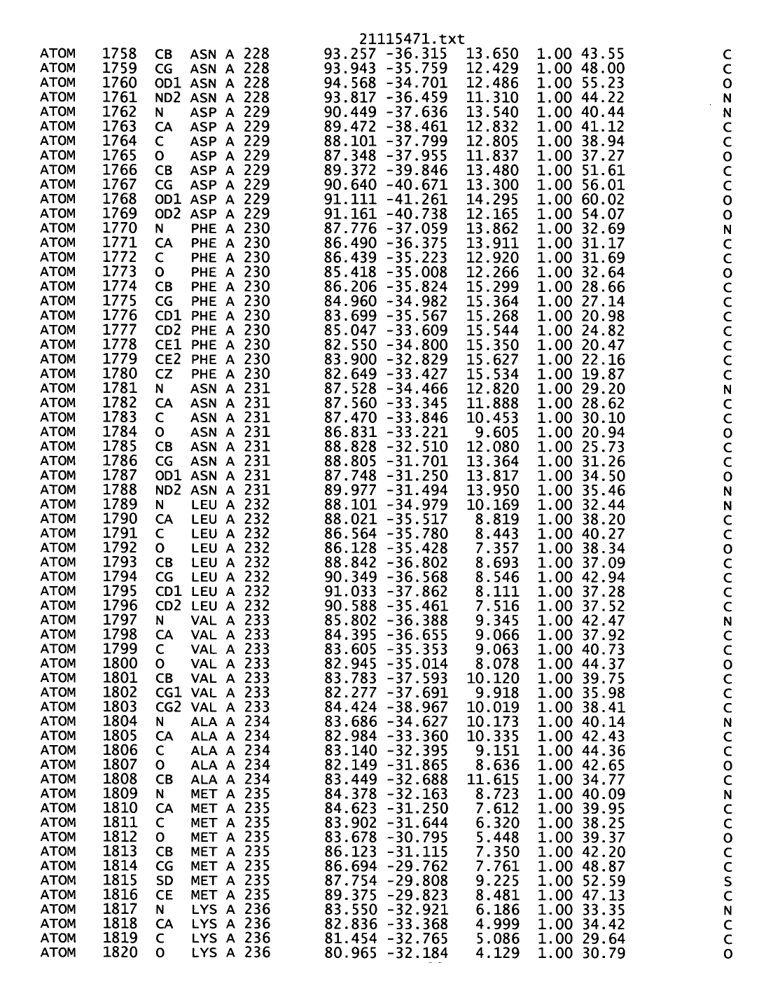

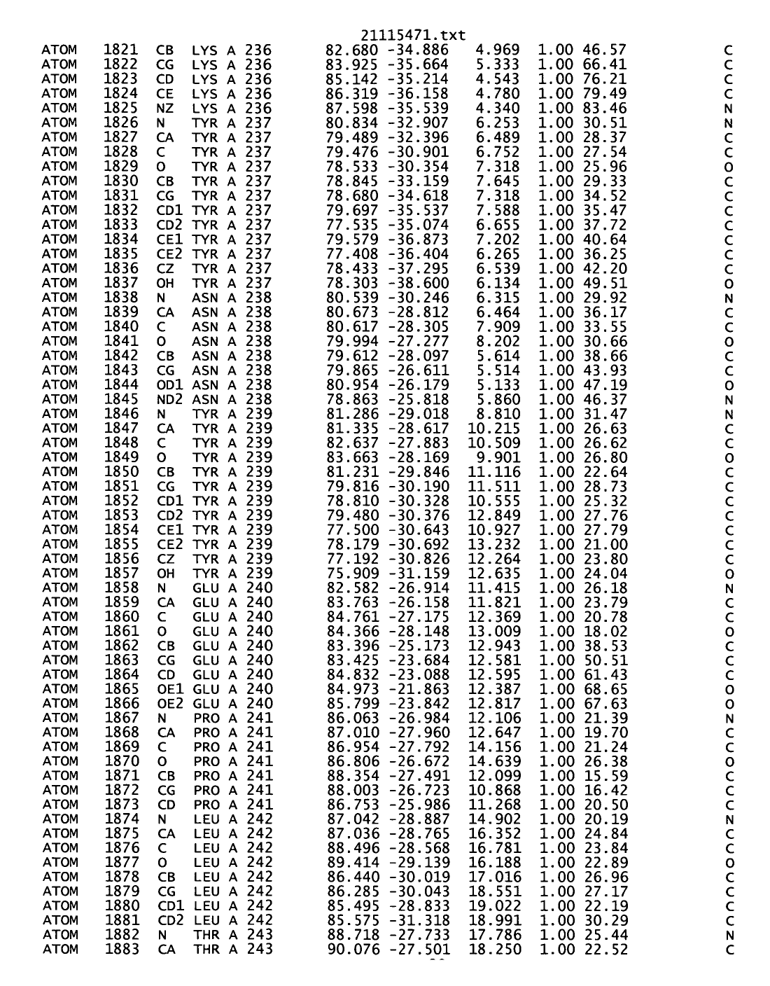

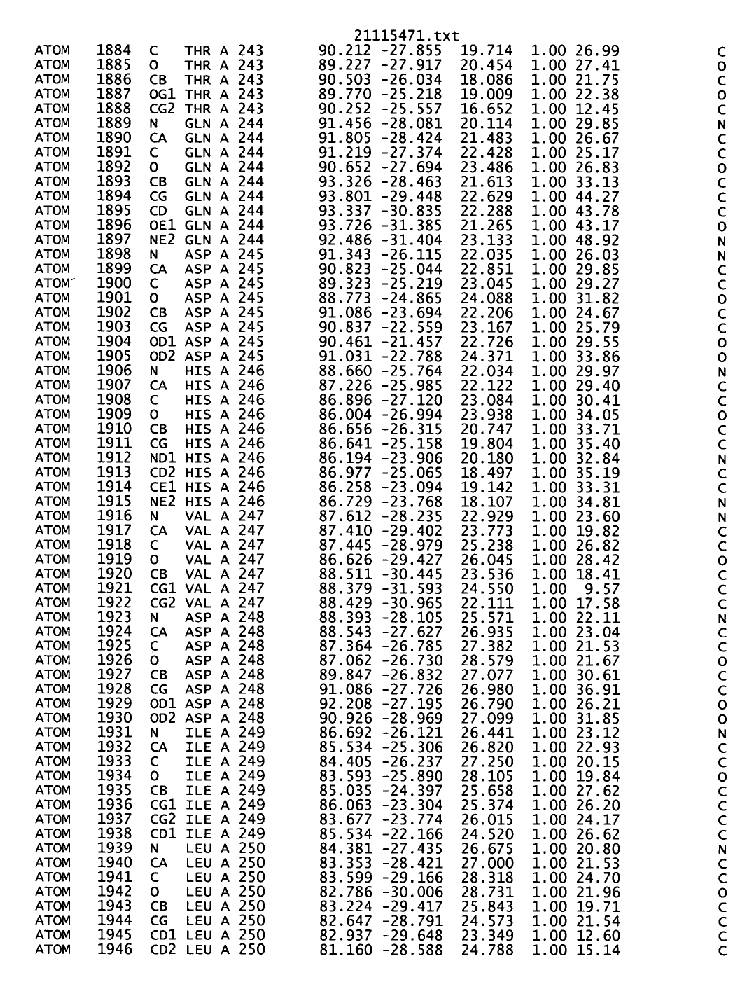

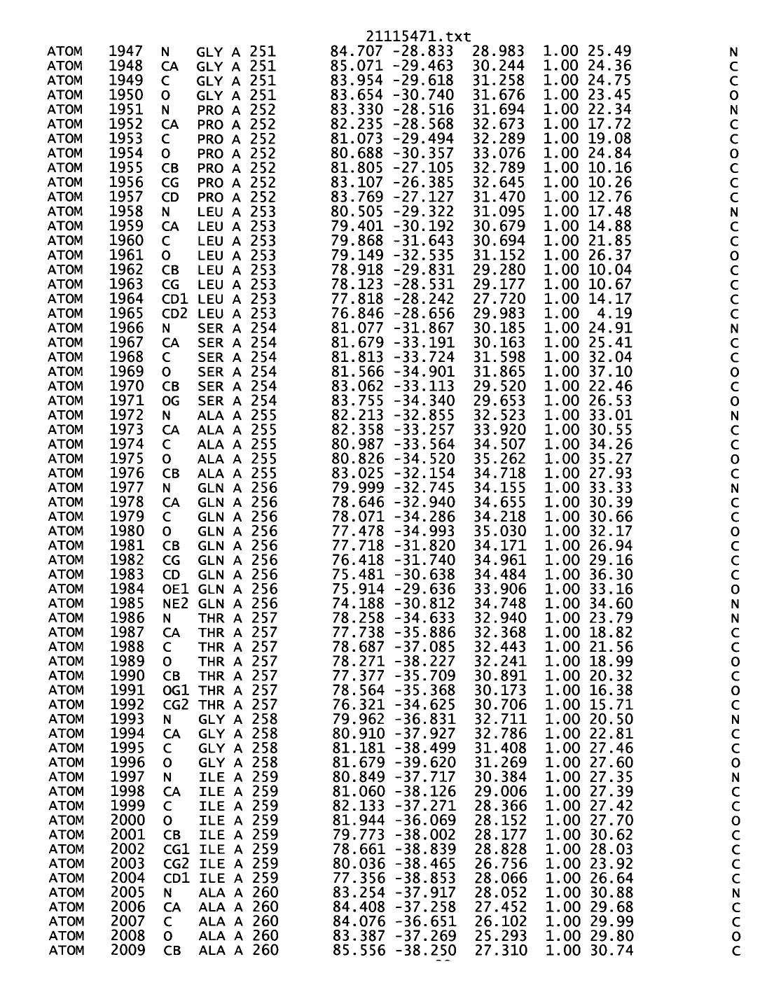

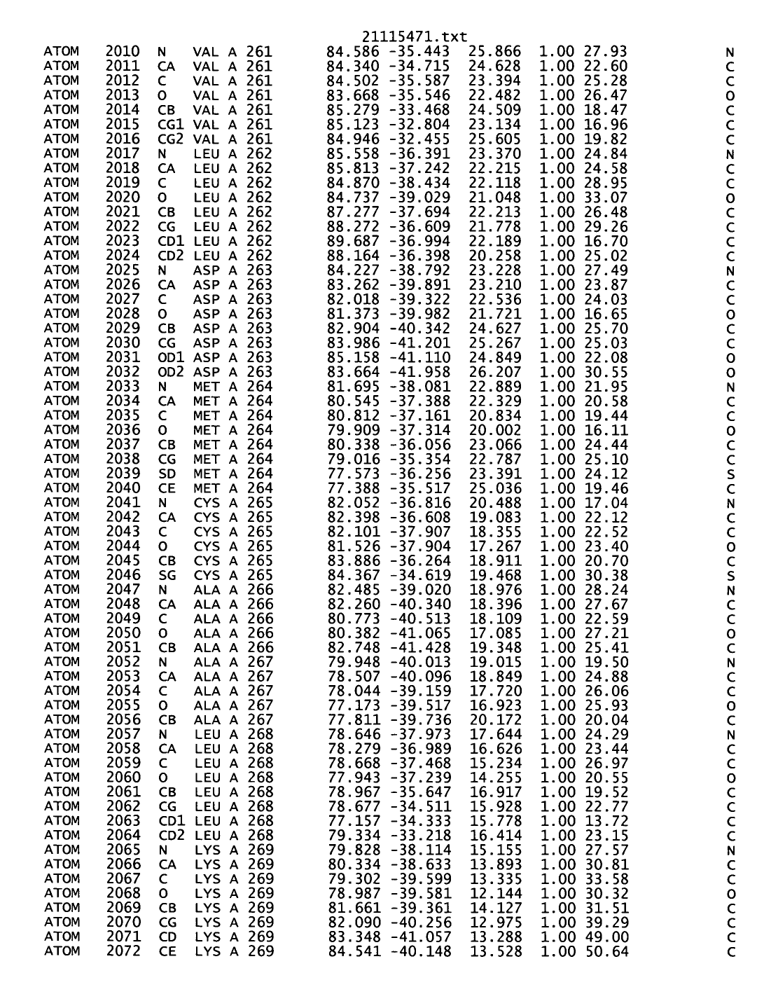

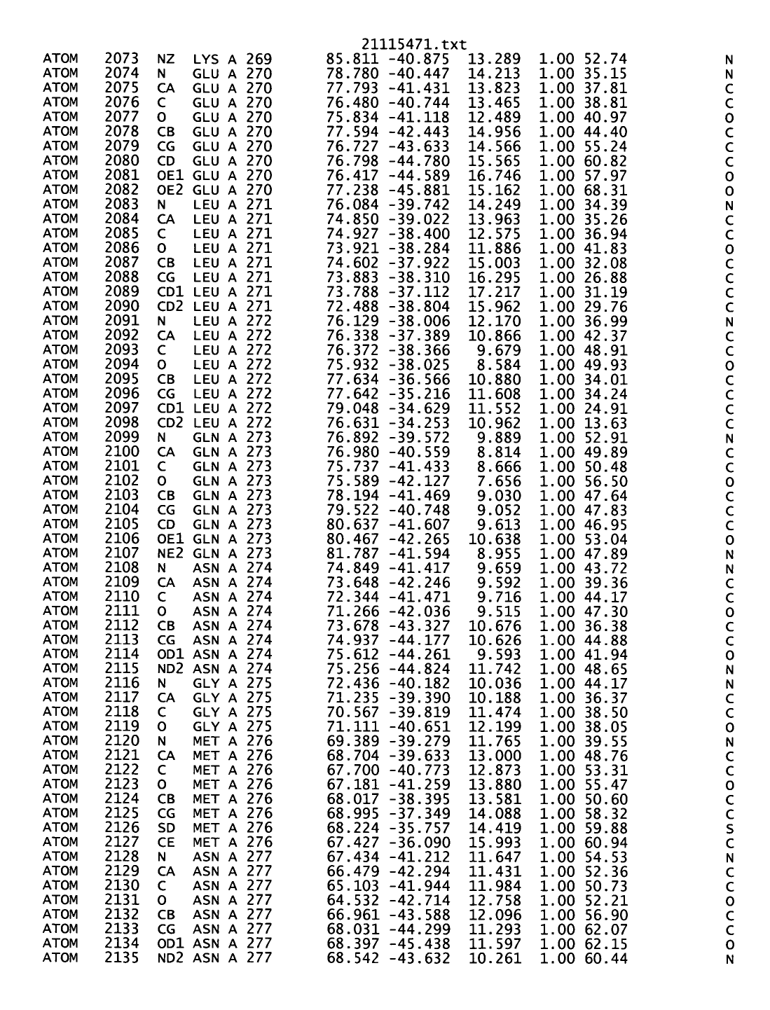

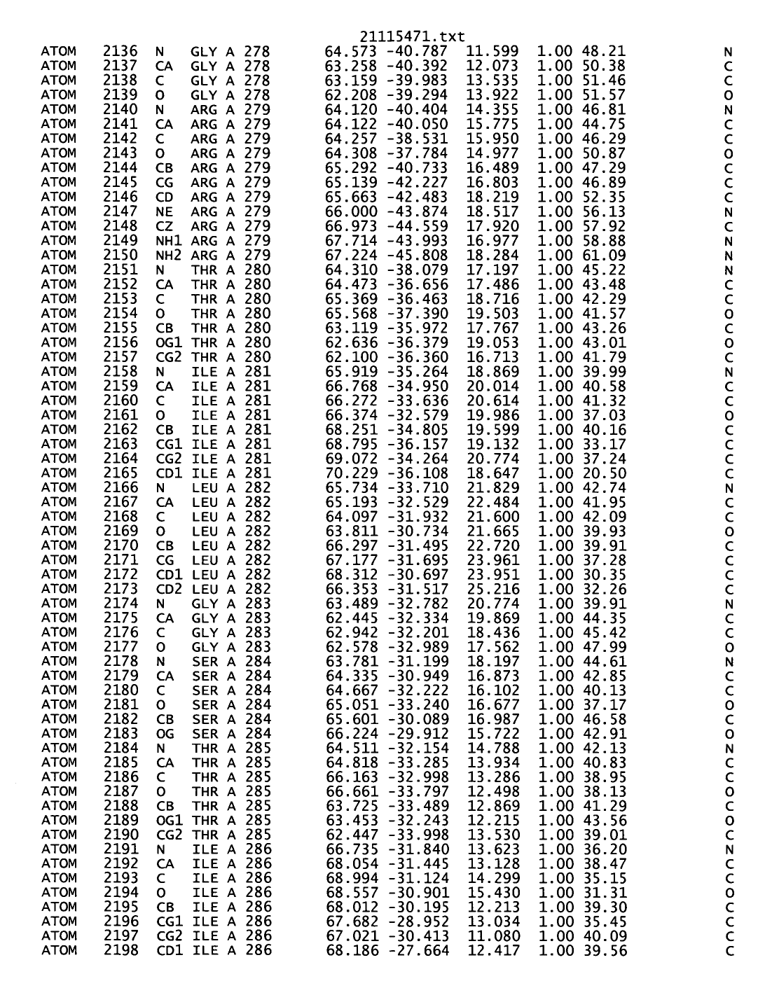

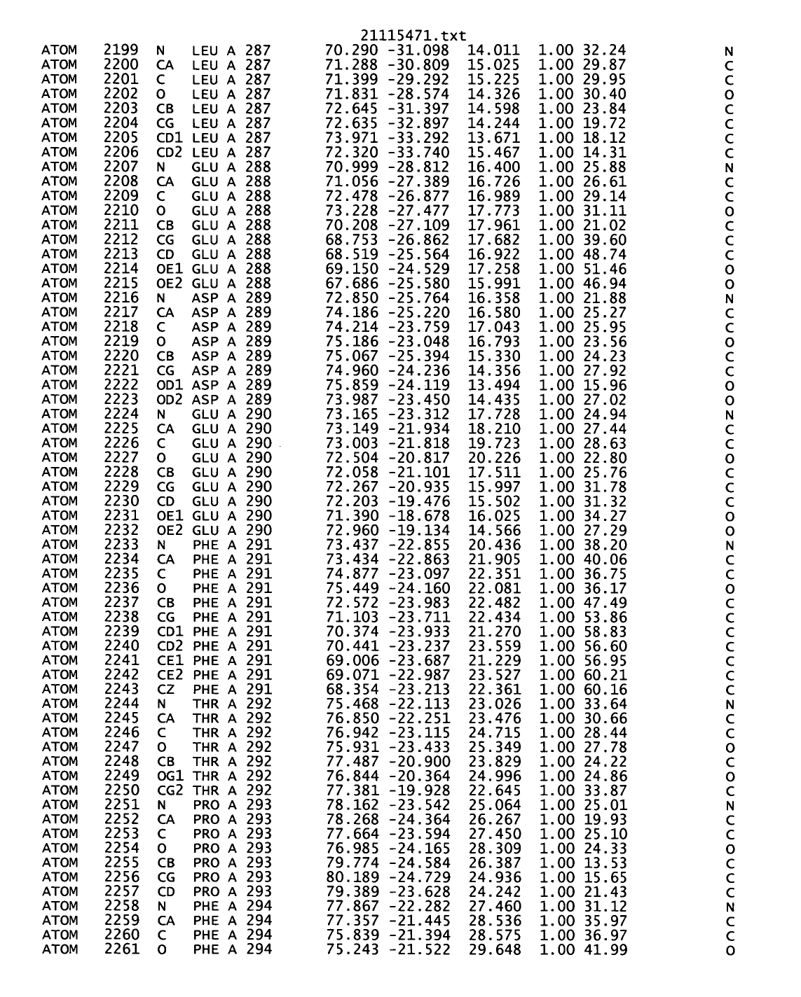

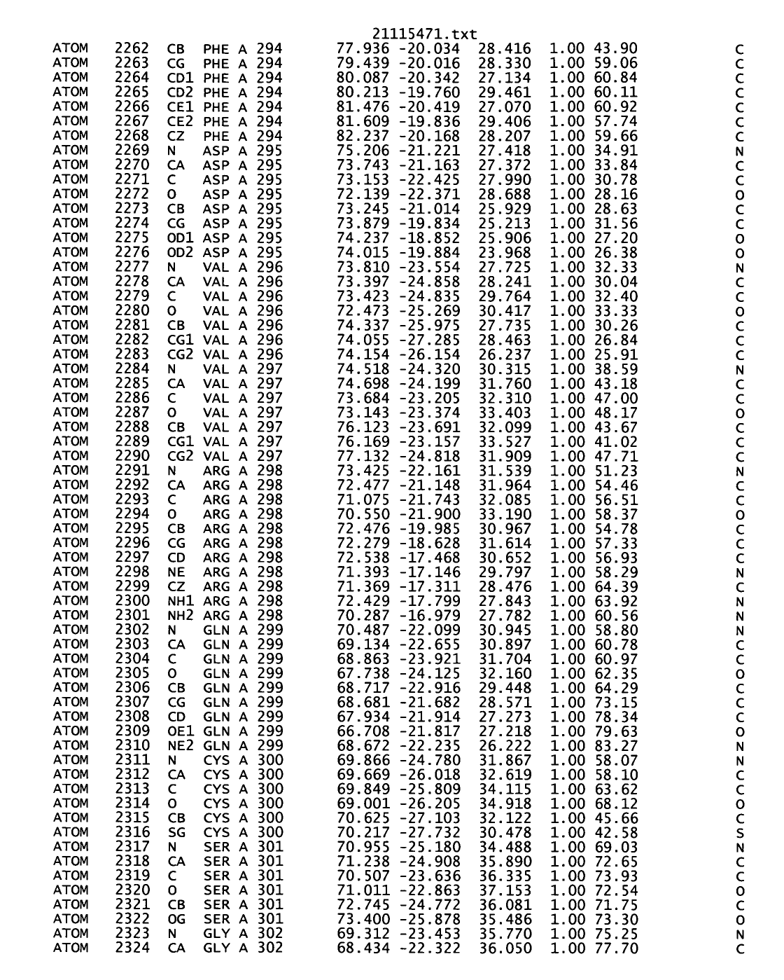

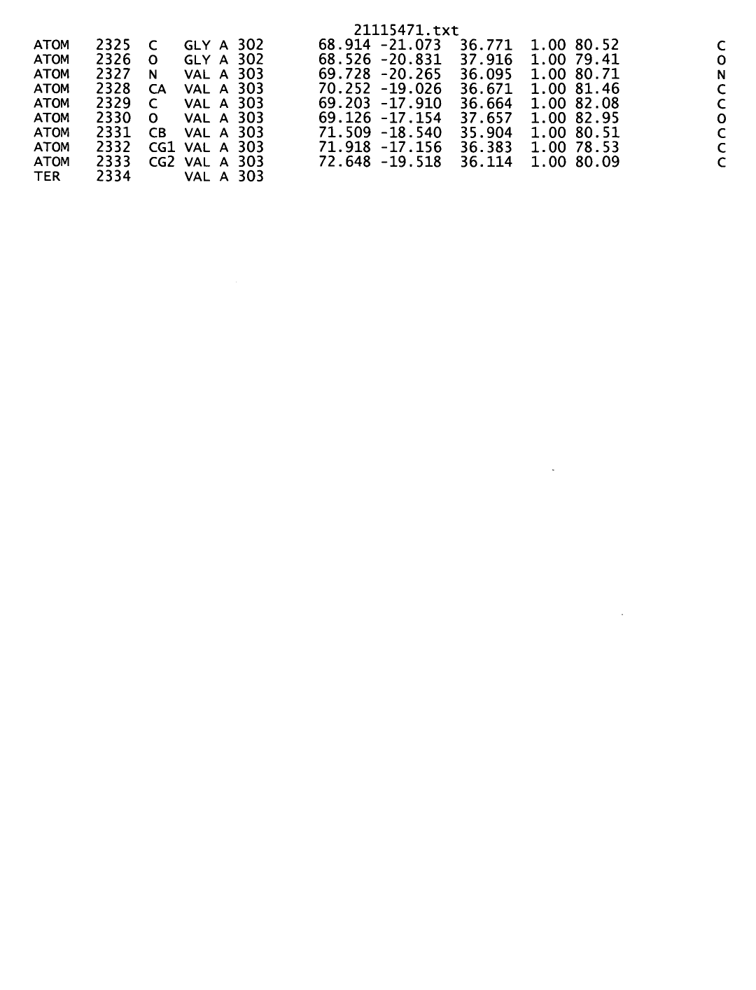

To judge whether a protein is an analogue of SARS CoV main protease, one can first define the atomic structure coordinates of the protein, i.e., the spatial positions of the atoms of the protein. The structure coordinates of a protein can be determined by X-ray or NMR. For example, diffraction data are obtained from mathematical equations related to the patterns obtained from diffraction of a monochromatic beam of X-rays by the atoms of the protein in crystal form, and then used to calculate electron density maps of the repeating unit of the crystal. The electron density maps can be used to establish the positions of the individual atoms of the protein. A program, Vector Alignment Search Tool (VAST), can be used to calculate the root square mean deviation between the protein and SARS Cov main protease. Specifically, the VAST program divides the protein into a number of fragments (e.g., 90-210 amino acid residues), compares the structure coordinates of the C-alpha atoms in these fragments with the coordinates of the C-alpha atoms of residue 1 to residue 189 of chain A of SASR CoV main protease (shown in FIG. 1), and provides a root square mean deviation. If the deviation is less than or equal to 2.3 Å, the protein is an analogue of SARS CoV main protease.

The above-described compounds modulate the activity of SARS CoV main protease and its analogues. These compounds can be used to treat diseases, such as coronavirus infection (including SARS virus infection and TGEV infection), hepatitis C, hemophilia, vascular restenosis, or hypertension. Thus, this invention features a method of treating coronavirus infection by administering an effective amount of a compound of Group V or VI, a method of treating hepatitis C by administering an effective amount of a compound of Group VII or VIII, a method of treating hemophilia by administering an effective amount of a compound of Group IX, and a method of treating vascular restenosis or hypertension by administering an effective amount of a compound of Group X. Each method may include administering an effective amount of another active agent. For example, an antiviral agent, such as interferon α or ribavirin, and a compound of Group VII or VIII can be used together to treat hepatitis C.

The term “treating” refers to administering the compound to a subject who has a disorder (i.e., coronavirus infection, hepatitis C virus infection, hemophilia, vascular restenosis, or hypertension), or has a symptom of the disorder, or has a predisposition toward the disorder, with the purpose to cure, heal, alleviate, relieve, alter, remedy, ameliorate, improve, or affect the disorder, the symptoms of the disorder, or the predisposition toward the disorder.

To practice the method of the present invention, a composition having one or more of the above-described compounds can be administered parenterally, orally, nasally, rectally, topically, or buccally. The composition may contain a pharmaceutical acceptable carrier. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol and water. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art. A composition having one or more active compounds can also be administered in the form of suppositories for rectal administration.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of a topical compound. Examples of other carriers include colloidal silicon oxide, magnesium stearate, cellulose, sodium lauryl sulfate, and D&C Yellow # 10.

The biological effects of the above-described compounds can be tested by an in vitro or in vivo assay. For example, compounds of Groups I, II, III, and IV can be preliminarily screened by in vitro assays in which the compounds are tested for their efficacy in modulating the activity of a protein, i.e., SARS CoV main protease or its analogue. Compounds that demonstrate high efficacy in the preliminary screening can be further evaluated by in vivo methods well known in the art to evaluate their activity in treating the disease in connection with the protein.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLE 1

### Inhibition HCV NS3 Main Protease Activity

Compound 1, 14, 20, 21, and 25 were purchased from Maybridge (England). A fluorogenic peptide substrate, Ac-ED(EDANS)EEAbuψ[COO]ASK(DABCYL), was synthesized and purified following the procedures described in Taliani, et. al. Analytical Biochemeistry, 1996, 240(1):60-67.

A protease domain of HCV NS3 (amino acids 1027-1206 of the HCV polyprotein) was prepared by the method described in Steinkuhler, et al. J. Biol. Chem. 1996, 271: 6367-6373. Briefly, NS3 was expressed in Escherichia coli using a T7 polymerase expression system and purified to homogeneity from the soluble fraction of E. coli BL21 (DE53) cell extract starting with a batchwise chromatography with SP-Sepharose resin (Pharmacia), followed by column chromatography on Superdex 75 (Pharmacia) and HR 5/5 Mono S (Pharmacia). Protein concentrations were estimated by the quantitative amino acid analysis or by determination of the absorbance at 280 nm using an extinction coefficient ε=18200 M⁻¹ cm⁻¹.

A continuous assay was performed on a fluorescence reader using black U-bottom 96-well plate at 23° C. following the procedure described in Taliani, et al. Analytical Biochemeistry, 1996, 240(1):60-67. Excitation was 355 nm and emission wavelength was 510 nm. The results show that all test compounds, i.e., compounds Compound 1, 14, 20, 21, and 25 effectively inhibited the proteolytic activity of HCV NS3 protease.

### EXAMPLE 2

### Inhibition of SARS CoV Main Protease Activity

Fluorogenic peptide substrate Dabcyl-KTSAVLQSGFRKME-Edans was obtained from Biogenesis (Taiwan). Compounds 1-21, 22, 25, and 26 were purchased from Maybridge (England).

Expression and purification of SARS CoV main protease were performed by the method described in Kuo, et al. Biochemical and Biophysical Research Communications, 2004, 318: 862-867.

A mixture containing 50 nM SARS protease, 6 μM fluorogenic peptide substrate in a buffer of 12 mM Tris-HCl (pH 7.5), 120 mM NaCl, 0.1 mM EDTA, and 1 mM DTT plus 7.5 mM b-ME was prepared. From this mixture, a series of solutions having different concentrations of a test compound (ranging from 0 to 50 μM) was obtained. The fluorescence change of the solutions was measured using a 96-well fluorescence plate reader. The initial velocities of the reactions were plotted against different compound concentrations to obtain the IC₅₀ values using the following equation:
A(I)=A(0)×{1−[I/(I+IC₅₀)]}
where A(I) is the enzyme activity at the compound concentration I; A(0) is the enzyme activity in the absence of the test compound; and I is the compound concentration.

The results show that all test compounds inhibit the proteolytic activity of SARS CoV main protease. Particularly, compounds 1-4 had very low IC₅₀ values

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. For example, compounds structurally analogous to above-described compounds also can be made, screened for the above-described activities and used to practice this invention. Thus, other embodiments are also within the claims.

What is claimed is:

1. A method for modulating activity of a protein, comprising contacting the protein with an effective amount of a compound of the following formula:
A₁-L-A₂
wherein
each of A₁ and A₂, independently, is phenyl, 5-membered heteroaryl, or 6-membered heteroaryl, optionally substituted with alkyl, haloalkyl, alkenyl, alkynyl, aryl, heteroaryl, halo, nitro, cyano, OR¹, OC(O)R¹, C(O)OR¹, C(O)R¹, SR¹, SO₂R¹, NR¹R², or NR¹C(O)R², or fused with a 3-8 membered ring; R¹ and R², independently, being H, alkyl, alkenyl, aryl, or heteroaryl; and
L is —SO₂—, —C(R³R⁴)SO₂—, —C(R³R⁴)NR⁵—, —C(O)—, —C(O)S—, —C≡C—, —C(R³R⁴)C(O)O—, or —S(O)₂NR³—; each of R³, R⁴, and R⁵, independently, being H, alkyl, aryl, or heteroaryl;
in which the protein is SARS CoV main protease or an analogue thereof.

2. The method of claim 1, wherein the protein is SARS CoV main protease, human coronavirus 229E main protease, TGEV main protease, human chymase, human neutrophil elastase, human cathepsin, HCV NS3 proteinase, streptomyces griseus proteinase B, human coagulation factor Xa, alpha chymotrypsin, factor B serine protease, or collagenase.

3. The method of claim 2, wherein the protein is SAR CoV main protease or HCV NS3 proteinase.

4. The method of claim 2, wherein A₁ is phenyl.

5. The method of claim 4, wherein L is —SO₂—.

6. The method of claim 5, wherein the protein is SARS CoV main protease or HCV NS3 proteinase.

7. The method of claim 6, wherein A₂ is phenyl.

8. The method of claim 6, wherein A₂ is pyrimidinyl or pyridinyl.

9. The method of claim 6, wherein A₂ is pyrazolyl.

10. The method of claim 1, wherein the compound inhibits activity of the protein.

11. A method for modulating activity of a protein, comprising contacting the protein with an effective amount of a compound of the following formula:
A₁-L-A₂
wherein
A₁ is 5-membered heteroaryl, or 3 to 8-membered heterocyclyl, optionally substituted with alkyl, haloalkyl, alkenyl, alkynyl, aryl, heteroaryl, halo, nitro, cyano, OR¹, OC(O)R¹, C(O)OR¹, C(O)R¹, SR¹, SO₂R¹, NR¹R², or NR¹C(O)R², or fused with a 3-8 membered ring; R¹ and R², independently, being H, alkyl, alkenyl, aryl, or heteroaryl;
A₂ is 5-membered heteroaryl, 6-membered heteroaryl, or 3 to 8-membered heterocyclyl, optionally substituted with alkyl, haloalkyl, alkenyl, alkynyl, aryl, heteroaryl, halo, nitro, cyano, OR³, OC(O)R³, C(O)OR³, C(O)R³, SR³, SO₂R³, NR³R⁴, or NR³C(O)R⁴, or fused with a 3 to 8-membered ring; R³ and R⁴, independently, being H, alkyl, alkenyl, aryl, or heteroaryl; and
L is —SO₂—, —C(R⁵R⁶)S—, —C(R⁵R⁶)NR⁷—, —C(O)O—, —C(R⁵R⁶)C(O)O—, —C(R⁵R⁶)SO₂—, —C(O)NR⁵—, —C(O)—, —C(O)(CR⁵R⁶)—, —C(O)S—, —C≡C—, —O—, —S—, —N—, —C(S)NR⁵, or —SO₂NR⁵—; each of R⁵, R⁶, and R⁷, independently, being H, alkyl, aryl, or heteroaryl;
in which the protein is SARS CoV main protease or an analogue thereof.

12. The method of claim 11, wherein the protein is SARS CoV main protease, human coronavirus 229E main protease, TGEV main protease, human chymase, human neutrophil elastase, human cathepsin, HCV NS3 proteinase, streptomyces griseus proteinase B, human coagulation factor Xa, alpha chymotrypsin, factor B serine protease, or collagenase.

13. The method of claim 12, wherein the compound inhibits activity of the protein.

14. The method of claim 12, wherein the protein is SARS CoV main protease or HCV NS3 proteinase.

15. The method of claim 12, wherein each of A₁ and A₂, independently, is 5-membered heteroaryl.

16. The method of claim 15, wherein each of A₁ and A₂, independently, is triazolyl, pyrazolyl, thienyl, isoxazolyl, thiazolyl, furyl, or [1,3,4]oxadiazolyl.

17. The method of claim 16, wherein the protein is SARS CoV main protease or HCV NS3 proteinase.

18. A method for modulating activity of a protein, comprising contacting the protein with an effective amount of a compound of the following formula:
A₁-L-A₂
wherein
A₁ is phenyl optionally substituted with alkyl, haloalkyl, alkenyl, alkynyl, aryl, heteroaryl, halo, nitro, cyano, OR¹, OC(O)R¹, C(O)OR¹, C(O)R¹, SR¹, SO₂R¹, NR¹R², or NR¹C(O)R², or fused with a 3-8 membered ring; R¹ and R², independently, being H, alkyl, alkenyl, aryl, or heteroaryl;
A₂ is 3 to 8-membered heterocyclyl, optionally substituted with alkyl, haloalkyl, alkenyl, alkynyl, aryl, heteroaryl, halo, nitro, cyano, OR³, OC(O)R³, C(O)OR³, C(O)R³, SR³, SO₂R³, NR³R⁴, or NR³C(O)R⁴, or fused with a 3 to 8-membered ring; R³ and R⁴, independently, being H, alkyl, alkenyl, aryl, or heteroaryl; and
L is deleted, —C(R⁵R⁶)S—, —C(R⁵R⁶)NR⁷—, —C(O)O—, —C(R⁵R⁶)C(O)O—, —C(R⁵R⁶)SO₂—, —C(O)NR⁵—, —C(O)—, —C(O)(CR⁵R⁶)—, —C(O)S—, —C≡C—, —O—, —S—, —N—, —C(S)NR⁵, or —SO₂NR⁵—; each of R⁵, R⁶, and R⁷, independently, being H, alkyl, aryl, or heteroaryl;
in which the protein is SARS CoV main protease or an analogue thereof.

19. The method of claim 18, wherein the protease is SARS CoV main protease, human coronavirus 229E main protease, TGEV main protease, human chymase, human neutrophil elastase, human cathepsin, HCV NS3 proteinase, streptomyces griseus proteinase B, human coagulation factor Xa, alpha chymotrypsin, factor B serine protease, or collagenase.

20. The method of claim 19, wherein the compound inhibits activity of the protein.

21. The method of claim 20, wherein the protein is SARS CoV main protease or HCV NS3 proteinase.

22. A method for modulating activity of a protein, comprising contacting the protein with an effective amount of a compound of the following formula:
A₁-L-A₂
wherein
each of A₁ and A₂, independently, is phenyl, or 5-membered heteroaryl, optionally substituted with alkyl, haloalkyl, alkenyl, alkynyl, aryl, heteroaryl, halo, nitro, cyano, OR¹, OC(O)R¹, C(O)OR¹, C(O)R¹, SR¹, SO₂R¹, NR¹R², or NR¹C(O)R², or fused with a 3-8 membered ring; R¹ and R², independently, being H, alkyl, alkenyl, aryl, or heteroaryl; and
L is deleted, —SO₂—, —C(R³R⁴)SO₂—, —C(R³R⁴)NR⁵—, —C(O)—, —C(O)S—, —C≡C—, —C(R³R⁴)C(O)O—, or —S(O)₂NR³—; each of R³, R⁴, and R⁵, independently, being H, alkyl, aryl, or heteroaryl;
in which the protein is SARS CoV main protease or an analogue thereof.

23. The method of claim 22, wherein the protease is SARS CoV main protease, human coronavirus 229E main protease, TGEV main protease, human chymase, human neutrophil elastase, human cathepsin, HCV NS3 proteinase, streptomyces griseus proteinase B, human coagulation factor Xa, alpha chymotrypsin, factor B serine protease, or collagenase.

24. The method of claim 22, wherein the compound inhibits activity of the protein.

25. The method of claim 22, wherein the protein is SARS CoV main protease or HCV NS3 proteinase.

26. A method for treating coronavirus infection, comprising administering to a subject in need thereof an effective amount of a compound of the following formula:
A₁-L-A₂
wherein
each of A₁ and A₂, independently, is phenyl, 5-membered heteroaryl, or 6-membered heteroaryl, optionally substituted with alkyl, haloalkyl, alkenyl, alkynyl, aryl, heteroaryl, halo, nitro, cyano, OR¹, OC(O)R¹, C(O)OR¹, C(O)R¹, SR¹, SO₂R¹, NR¹R², or NR¹C(O)R², or fused with a 3-8 membered ring; R¹ and R², independently, being H, alkyl, alkenyl, aryl, or heteroaryl; and
L is deleted, —SO₂—, —C(R³R⁴)SO₂—, —C(R³R⁴)NR⁵—, —C(O)—, —C(O)S—, —C≡C—, —C(R³R⁴)C(O)O—, or —S(O)₂NR³—; each of R³, R⁴, and R⁵, independently, being H, alkyl, aryl, or heteroaryl.

27. The method of claim 26, wherein the coronavirus infection is severe acute respiratory syndrome virus infection.

28. The method of claim 27, wherein A₁ is phenyl.

29. The method of claim 28, wherein L is —SO₂—.

30. The method of claim 29, wherein the coronavirus infection is severe acute respiratory syndrome virus infection.

31. The method of claim 30, wherein A₂ is phenyl.

32. The method of claim 30, wherein A₂ is pyrimidinyl, pyrazolyl, or pyridinyl.

33. A method for treating coronavirus infection, comprising administering to a subject in need thereof an effective amount of a compound of the following formula:
A₁-L-A₂
wherein
A₁ is phenyl, 5-membered heteroaryl, or 3 to 8-membered heterocyclyl, optionally substituted with alkyl, haloalkyl, alkenyl, alkynyl, aryl, heteroaryl, halo, nitro, cyano, OR¹, OC(O)R¹, C(O)OR¹, C(O)R¹, SR¹, SO₂R¹, NR¹R², or NR¹C(O)R², or fused with a 3-8 membered ring; R¹ and R², independently, being H, alkyl, alkenyl, aryl, or heteroaryl;
A₂ is 5-membered heteroaryl, 6-membered heteroaryl, or 3 to 8-membered heterocyclyl, optionally substituted with alkyl, haloalkyl, alkenyl, alkynyl, aryl, heteroaryl, halo, nitro, cyano, OR³, OC(O)R³, C(O)OR³, C(O)R³, SR³, SO₂R³, NR³R⁴, or NR³C(O)R⁴, or fused with a 3 to 8-membered ring; R³ and R⁴, independently, being H, alkyl, alkenyl, aryl, or heteroaryl; and
L is deleted, —SO₂—, —C(R⁵R⁶)S—, —C(R⁵R⁶)NR⁷—, —C(O)O—, —C(R⁵R⁶)C(O)O—, —C(R⁵R⁶)SO₂—, —C(O)NR⁵—, —C(O)—, —C(O)(CR⁵R⁶)—, —C(O)S—, —C≡C—, —O—, —S—, —N—, or —SO₂NR⁵—; each of R⁵, R⁶, and R⁷, independently, being H, alkyl, aryl, or heteroaryl.

34. The method of claim 33, wherein the coronavirus infection is severe acute respiratory syndrome virus infection.

35. The method of claim 34, wherein each of A₁ and A₂, independently, is 5-membered heteroaryl.

36. The method of claim 35, wherein each of A₁ and A₂, independently, is triazolyl, pyrazolyl, thienyl, isoxazolyl, thiazolyl, furyl, or [1,3,4]oxadiazolyl.

37. A method for treating hepatitis C virus infection, comprising administering to a subject in need thereof an effective amount of a compound of the following formula:
A₁-L-A₂
wherein
each of A₁ and A₂, independently, is phenyl, 5-membered hetereoaryl, 6-membered heteroaryl, or 3 to 8-membered heterocyclyl, each of which is optionally substituted with alkyl, haloalkyl, alkenyl, alkynyl, aryl, heteroaryl, halo, carboxy, acylalkyl, nitro, cyano, OR¹, OC(O)R¹, C(O)OR¹, C(O)R¹, SR¹, SO₂R¹, NR¹R², or NR¹C(O)R², or fused with a 3-8 membered ring; R¹ and R², independently, being H, alkyl, alkenyl, aryl, or heteroaryl; and
L is —SO₂—, —C(R³R⁴)S—, —C(R³R⁴)NR⁵—, —C(O)O—, —C(R³R⁴)C(O)O—, —C(R³R⁴)SO₂—, —C(O)NR³—, —C(O)—, —C(O)(CR³R⁴)—, —C(O)S—, —C≡C—, —O—, —S—, —N—, —C(S)NR³, or —SO₂NR⁵—; each of R³, R⁴, and R⁵, independently, being H, alkyl, aryl, or heteroaryl.

38. A method for treating hepatitis C virus infection, comprising administering to a subject in need thereof an effective amount of a compound of the following formula:
A₁-L-A₂
wherein
each of A₁ and A₂, independently, is phenyl, 5-membered hetereoaryl, or 3 to 8-membered heterocyclyl, each of which is optionally substituted with alkyl, haloalkyl, alkenyl, alkynyl, aryl, heteroaryl, halo, carboxy, acylalkyl, nitro, cyano, OR¹, OC(O)R¹, C(O)OR¹, C(O)R¹, SR¹, SO₂R¹, NR¹R², or NR¹C(O)R², or fused with a 3-8 membered ring; R¹ and R², independently, being H, alkyl, alkenyl, aryl, or heteroaryl; and
L is deleted, —SO₂—, —C(R³R⁴)S—, —C(R³R⁴)NR⁵—, —C(O)O—, —C(R³R⁴)C(O)O—, —C(R³R⁴)SO₂—, —C(O)NR³—, —C(O)—, —C(O)(CR³R⁴)—, —C(O)S—, —C≡C—, —O—, —S—, —N—, —C(S)NR³, or —SO₂NR⁵—; each of R³, R⁴, and R⁵, independently, being H, alkyl, aryl, or heteroaryl.
